(19) United States
(12) Patent Application Publication
(10) Pub. No.: US 2011/0207660 A1
(43) Pub. Date: Aug. 25, 2011

(54) PHARMACEUTICAL FORMULATIONS OF AN HCV PROTEASE INHIBITOR IN A SOLID MOLECULAR DISPERSION

(75) Inventors: Ashlesh Sheth, Basking Ridge, NJ (US); Chengjiu Hu, Morris Plains, NJ (US); Baohua Yue, Ballwin, MO (US); Marcelo Osvaldo Omelczuk, Austin, TX (US)

(73) Assignee: Schering Corporation, Kenilworth, NJ (US)

(21) Appl. No.: 13/057,339

(22) PCT Filed: Aug. 7, 2009
(86) PCT No.: PCT/US2009/053076

§ 371 Date: Apr. 11, 2011

Related U.S. Application Data
(60) Provisional application No. 61/086,997, filed on Aug. 7, 2008.

(51) Int. Cl. A61K 38/06; A61P 31/14; A61P 1/16

(52) U.S. Cl. 514/4.3; 514/21.9

(57) ABSTRACT

The present invention provides pharmaceutical formulations of an HCV protease inhibitor in a solid dispersion with an excipient which provided advantageous pharmacokinetic properties for inhibiting or treating HCV infection. In preferred embodiments, the excipient is at least one polymer. The present invention also provides processes for manufacturing such formulations as well as uses of said composition for the manufacture of a medicament for treating or ameliorating one or more symptoms of HCV or disorders associated with HCV in a subject in need thereof using said formulations.

### FIELD OF THE INVENTION

The present invention relates to novel pharmaceutical formulations comprising a hepatitis C virus (HCV) protease inhibitor in a solid molecular dispersion with an excipient, said excipient comprising preferably at least one polymer. The invention also relates to processes for manufacturing such formulations as well as methods for treating or ameliorating one or more symptoms of HCV or disorders associated with HCV in a subject in need thereof using said formulations.

### BACKGROUND OF THE INVENTION

Citation of or reference to any application or publication in this Section or any Section of this application is not an admission that such document is available as prior art to the present invention.

HCV infection, implicated in cirrhosis of the liver and in induction of hepatocellular carcinoma, is more difficult to treat than other forms of hepatitis due to the lack of immunity or remission associated with HCV infection. Patients suffering from HCV infection face a poor prognosis with approximately 50% failing to respond to the current standard of care, that is, pegylated interferon or pegylated interferon/ribavirin combination therapy. Generally, patients infected with HCV genotype 1, the most common subtype of HCV in North America and Europe, fail to respond to such therapies. Moreover, these therapies are expensive, often poorly tolerated, and unsuitable for certain patient populations. Thus, there remains an urgent unmet medical need to offer new therapies for HCV infected patients.

HCV protease inhibitors and methods of making the same, including the compound having the following chemical structure:

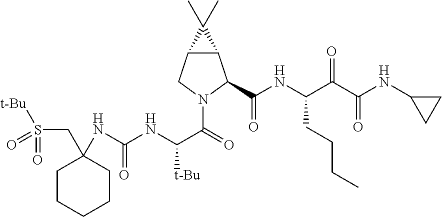

(referred to herein as Compound I) or a solvate thereof, are described in International Patent Publication WO2005/087731 (see, e.g., page 299, Example 792 to page 355, Example 833) the entire disclosure of which is incorporated herein by reference. International Patent Publication WO2005/087731 also generally describes pharmaceutical compositions of HCV protease inhibitors, including Compound I or a solvate thereof. U.S. Patent Publication Nos. 2006/0275366 and 2007/0237818 describe controlled-release pharmaceutical compositions of HCV protease inhibitors, including Compound I or a solvate thereof. U.S. Patent Publication No. 2007/0010431 describes pharmaceutical compositions of HCV protease inhibitors, including Compound I or a solvate thereof, with at least one surfactant. U.S. Patent Publication No. 2007/0287664 generally describes administration of HCV protease inhibitors, including Compound I or a solvate thereof, in combination with at least one cytochrome P450 isoenzyme 3A4 (CYP3A4) inhibitor. U.S. Patent Publication Nos. 2006/0275366, 2007/0237818, 2007/0010431, and 2007/0287664 also describe methods of using the compositions described therein to treat HCV infection in a subject in need thereof.

The development of commercially suitable pharmaceutical formulations of Compound I or a solvate thereof necessitates overcoming multiple physicochemical and pharmacokinetic challenges. Notably, Compound I is susceptible to epimerization (to an inactive form of Compound I), oxidation, and hydrolysis. In addition, according to the Biopharmaceutics Classification System, Compound I is a Class IV compound, that is, a compound having low solubility and low permeability. Consequently, Compound I has relatively low bioavailability. Thus, pharmaceutical formulations of Compound I or a solvate thereof are needed that provide acceptable drug loading, dissolution, stability, and bioavailability for a treatment regimen wherein the number of doses administered per day to achieve the desired therapeutic plasma concentration could be reduced. Such formulations would reduce the dose, reduce the cost of goods for the product, and/or reduce the dosing regimen. Such pharmaceutical formulations would also provide greater convenience for patients and hence promote patient compliance thereby reducing the potential for development of drug-resistant HCV strains. These and other objectives are provided by the novel pharmaceutical formulations and processes of the present invention.

### SUMMARY OF THE INVENTION

The pharmaceutical formulations of the present invention address, inter alia, the aforementioned needs. In particular, pharmaceutical formulations of the present invention provide enhanced bioavailability of Compound I compared to pharmaceutical formulations in which micronized or amorphous Compound I is blended with sodium lauryl sulfate. Surprisingly, pharmaceutical formulations of the present invention also provide a favorable pharmacokinetic profile in humans for Compound I, a BCS class IV compound. In fact, the pharmaceutical formulations of the present invention provide sufficient bioavailability when administered in a once-a-day (QD) or twice-a-day (BID) dosing regimen in combination with a cytochrome P450 inhibitor to achieve the desired therapeutic plasma concentration of Compound I. Additionally, the pharmaceutical formulations of the present invention provide sufficient bioavailability when administered in a thrice-a-day (TID) dosing regimen alone (i.e., without administration of a cytochrome P450 inhibitor). Furthermore, the pharmaceutical formulations of the present invention provide a commercially acceptable shelf-life projected to be at least 1 year under ambient conditions. In fact, it has been surprisingly found that the present formulations comprising an intimate molecular dispersion of Compound 1 and an excipient, preferably a non-swellable polymer are more stable than Compound 1 alone.

The present invention provides a pharmaceutical formulation comprising: (a) Compound I; and (b) an excipient; wherein (a) and (b) are in a solid molecular dispersion. In preferred embodiments, the excipient is at least one polymer. According to the present invention, Compound 1 in a stable amorphous form is uniformly dispersed in a polymer. The solid dispersions exhibit excellent mechanical and physical attributes necessary for subsequent roller compaction, milling, blending, and tablet compression. In certain embodiments, the formulations of the present invention may optionally further comprise one or more additional pharmaceutically acceptable excipients. The solid dispersions of the present invention can be directly utilized as pharmaceutical formulations (e.g., powders or granules). Alternatively, such solid dispersions can be used to prepare pharmaceutical formulations in other forms including capsules, tablets, and unit dose packets. In fact, the solid dispersions provided herein are suitable for high drug loading dosage forms with ≧100 mg drug per unit dosage form.

In one embodiment, at least one polymer is carbomer, a polymer of acrylic acid), cellulose acetate phthalate, hydroxypropyl cellulose, hydroxyethyl cellulose, hydroxypropylmethylcellulose, hydroxypropyl methylcellulose phthalate, polyacrylate polymer, polyethylene oxide, polyvinyl alcohol, poloxamer, povidone, polytheylene glycol, copovidone, or hypromellose acetate succinate (hydroxypropyl methylcellulose acetate succinate; HPMCAS), or a combination of two or more thereof. In certain preferred embodiments, at least one polymer is poloxamer, povidone, polytheylene glycol, copovidone, hydroxypropylmethylcellulose, or hypromellose acetate succinate, or a combination of two or more thereof. In one preferred embodiment, at least one polymer is copovidone. Polymers used as a solid dispersion agent may make up about 5% to about 95% by weight of the pharmaceutical formulation. In certain embodiments, polymer used as a solid dispersion agent is present at about 10% to about 90% by weight of the pharmaceutical formulation. In one preferred embodiment, polymer used as a solid dispersion agent is present at about 20% to about 80% by weight of the pharmaceutical formulation.

In certain embodiments, the ratio by weight of (a) to (b) is in the range of about 10:1 to about 1:10. In certain preferred embodiments, the ratio by weight of (a) to (b) is in the range of about 2:1 to about 1:4, more preferably about 1:1 to about 1:3. In one preferred embodiment, the ratio by weight of (a) to (b) is about 1:1. In another preferred embodiment, the ratio by weight of (a) to (b) is about 1:3. In certain embodiments, the pharmaceutical formulation further comprises one or more additional pharmaceutically acceptable excipients. In one preferred embodiment, the pharmaceutical formulation further comprises a lubricant. In another preferred embodiment, the pharmaceutical formulation further comprises stearic acid, magnesium stearate, calcium stearate, fat, wax, hydrogenated vegetable oil, castor oil, glycerin monostearate, glyceryl behenate, sodium stearyl fumurate, zinc stearate, glyceryl palmitostearate, medium-chain triglyceride, or mineral oil, or a combination of two or more thereof. In certain embodiments, the pharmaceutical formulation further comprises a diluent, a disintegrant, a surfactant, a glidant, and/or a lubricant, or a combination of two or more thereof.

In certain embodiments, Compound I in an amorphous form is stable within the solid dispersion of the invention after storage at 40° C. and 75% relative humidity for at least 3 months.

In certain embodiments, the pharmaceutical formulation of the invention provides release of at least about 75% Compound I in 45 minutes when tested using a USP Dissolution Apparatus II with a paddle operated at 75 RPM filled with 900 mL of dissolution medium at pH 3.5 comprising 0.5% sodium lauryl sulfate in 0.05% acetic acid maintained at 37° C.±0.5° C.

The present invention also provides methods for treating or ameliorating one or more symptoms of HCV or disorders associated with HCV, comprising the step of administering to a patient in need thereof a pharmaceutical formulation comprising: (a) Compound I; and (b) at least one excipient, preferably one polymer; wherein (a) and (b) are in a solid molecular dispersion.

In certain embodiments, pharmaceutical formulations of the present invention are administered once-a-day (QD), twice-a-day (BID), or thrice-a-day (TID). A typical recommended daily dosage regimen for treating or ameliorating one or more symptoms of HCV or disorders associated with HCV in a subject in need thereof can range from about 100 mg/day to about 4800 mg/day Compound I. In certain preferred embodiments, the recommended daily dosage regimen for treating or ameliorating one or more symptoms of HCV or disorders associated with HCV in a subject in need thereof can range from about 600 mg TID to about 1600 mg TID Compound I. Such TID dosage regimens can be administered in the absence of a cytochrome P450 inhibitor. In other embodiments, the pharmaceutical formulations of the present invention are administered in combination with a cytochrome P450 inhibitor, preferably a CYP3A4 inhibitor (e.g., ritonavir, preferably at a dose of 100 mg ritonavir administered either QD or BID).

The recommended daily dosage regimen for treating or ameliorating one or more symptoms of HCV or disorders associated with HCV in a subject in need thereof can range from about 100 mg DID to about 400 mg BID Compound I in a novel formulation of the present invention in combination with a cytochrome P450 inhibitor (e.g., about 100 mg ritonavir BID). In yet other embodiments, the recommended daily dosage regimen for treating or ameliorating one or more symptoms of HCV or disorders associated with HCV in a subject in need thereof can range from about 100 mg QD to about 600 mg QD Compound I in combination with a cytochrome P450 inhibitor (e.g., about 100 mg ritonavir QD).

The present invention also provides robust manufacturing processes that allow novel pharmaceutical formulations of the present invention to be readily and reliably prepared with satisfactory processability for commercialization. In preferred embodiments, the present invention provides methods for preparing a pharmaceutical formulation comprising Compound I in a solid dispersion with at least one excipient, preferably a polymer, comprising the steps of (a) dissolving Compound I or a solvate thereof and at least one excipient, preferably a polymer in an organic solvent; and (b) evaporating the organic solvent. As a starting material, Compound I can be in crystalline or amorphous form. In certain embodiments, the dissolving step is performed at a temperature in the range of about 5° C. to about 70° C. In certain embodiments, the evaporating step is performed at a temperature in the range of about 20° C. to about 80° C. In certain embodiments, the organic solvent is ethanol, methanol, acetone, methylenechloride, dichloromethane, ethyl acetate, water, chloroform, toluene, or a combination of two or more thereof. According to the present invention, dissolving Compound I or a solvate thereof and at least one excipient, preferably a polymer, in an organic solvent and then evaporating the solvent forms an intimate molecular dispersion of Compound 1 in an amorphous form with the excipient, preferably a non-swellable polymer, which dispersion has surprisingly robust stability and characteristics amenable to tablet formation. The dispersions are substantially free (i.e. contain ≦2%, ≦3%, or ≦5%) of crystalline (or solvated) form of Compound I.

In one aspect the present invention provides pharmaceutical formulations comprising Compound I and at least one excipient, preferably a polymer in a solid dispersion which provides a mean steady-state AUC of Compound I that is about 21,000 hr-ng/ml when administered at a dose equivalent to 300 mg Compound I in combination with 100 mg ritonavir once-a-day to a patient. The present invention also encompasses pharmaceutical formulations which are similarly bioavailable such that the relative mean steady-state AUC of Compound I is within 80% to 125% of 21,000 hr-ng/ml, that is within the range from about 16,800 ng-hr/ml to about 26,250 hr-ng/ml, when administered at a dose equivalent to 300 mg Compound I in combination with 100 mg ritonavir once-a-day to a patient. In one embodiment, the pharmaceutical formulation provides a mean steady-state AUC of Compound I which is at least 80% of 21,000 hr-ng/ml, that is at least 16,800 hr-ng/ml, when administered at a dose equivalent to 300 mg Compound I in combination with 100 mg ritonavir once-a-day to a patient. In a certain embodiments, the pharmaceutical formulations provide a mean steady-state AUC of Compound I which is at least 21,000 hr-ng/ml when administered at a dose equivalent to 300 mg Compound I in combination with 100 mg ritonavir once-a-day to a patient.

In another aspect the present invention provides pharmaceutical formulations comprising Compound I in a solid dispersion which provides a mean steady-state Cmin of Compound I that is at least 200 ng/ml when administered at a dose equivalent to 300 mg Compound I in combination with 100 mg ritonavir once-a-day to a patient.

In one embodiment, the pharmaceutical formulation provides a mean steady-state Cmax of Compound I that is at least 2216 ng/ml when administered at a dose equivalent to 300 mg Compound I in combination with 100 mg ritonavir once-a-day to a patient. The mean Tmax is in the range from about 2 hours to about 6 hours post-dose.

In one embodiment, the pharmaceutical formulation provides a mean steady-state Cmax of Compound I that is about 2770 ng/ml when administered at a dose equivalent to 300 mg Compound I in combination with 100 mg ritonavir once-a-day to a patient. The present invention also encompasses pharmaceutical formulations which are similarly bioavailable such that the relative mean steady-state Cmax of Compound I is within 80% to 125% of 2770 ng/ml, that is within the range from about 2216 ng/ml to about 3463 ng/ml, when administered at a dose equivalent to 300 mg Compound I in combination with 100 mg ritonavir once-a-day to a patient. In one embodiment, the pharmaceutical formulation provides 2216 ng/ml when administered at a dose equivalent to 300 mg Compound I in combination with 100 mg ritonavir once-a-day to a patient. In a certain embodiment, the pharmaceutical formulation provides a mean steady-state AUC of Compound I which is at least 2770 ng/ml when administered at a dose equivalent to 300 mg Compound I in combination with 100 mg ritonavir once-a-day to a patient.

In certain preferred embodiments, the amount of Compound I is equivalent to 300 mg Compound I.

The present invention also provides preferred pharmaceutical formulations comprising Compound I and at least one polymer in a solid dispersion which provides a mean steady-state AUC of Compound I that is at least 16800 hr-ng/ml when administered at a dose equivalent to 300 mg Compound I in combination with a cytochrome P450 inhibitor once-a-day to a patient.

In another aspect the present invention provides preferred pharmaceutical formulations comprising Compound I and at least one polymer in a solid dispersion which provides a mean steady-state Cmin of Compound I that is at least 200 ng/ml when administered at a dose equivalent to 300 mg Compound I in combination with a cytochrome P450 inhibitor once-a-day to a patient.

In certain embodiments, the pharmaceutical formulation provides a mean steady-state Cmax of Compound I that is at least 2216 ng/ml when administered at a dose equivalent to 300 mg Compound I in combination with a cytochrome P450 inhibitor once-a-day to a patient. In certain embodiments, the pharmaceutical formulation provides a mean Tmax that is in the range from about 0.5 hour to about 6 hours.

In certain embodiments, the cytochrome P450 inhibitor is a cytochrome P450 isoenzyme 3A4 inhibitor. In certain embodiments, the cytochrome P450 inhibitor is ritonavir. In one embodiment, ritonavir is administered at a dose of 100 mg once-a-day. In another embodiment, ritonavir is administered at a dose of 100 mg twice-a-day.

### BRIEF DESCRIPTION OF THE DRAWINGS

FIG. 1 is a graph of the mean plasma concentration/time profile of Compound I following a single oral administration of 200 mg Compound I in various comparative formulations (1-3) and of exemplary formulations R and S of the present invention to dogs under fasted conditions. (For details, see Example 1, infra, especially Table 3B for exemplary formulations R and S of the present invention; see Example 2, infra, for comparative formulations 1-3 Table 5A.

FIG. 2 is a graph of the mean plasma concentration/time profile of Compound I following a single oral administration of 400 mg Compound I in a comparative formulation (8) and exemplary formulations of the present invention F and T in tablet or capsule forms to dogs under fasted conditions. For details, see Example 1, especially Tables 1B and 3B, respectively, for exemplary formulations F and T of the invention; see Example 2 especially Table 5B for comparative formulation 8.

FIG. 3 is a graph of the mean plasma concentration/time profile of Compound I following a single oral administration of a formulation of the present invention (exemplary formulation G) in a dose of 200 mg Compound I (in either capsule or tablet form) or as a comparative example (i.e. a suspension) to healthy human subjects under fed conditions. See Example 3, infra, for details.

FIG. 4 is a graph of the mean plasma concentration/time profile of Compound I following a single oral administration of a formulation of the present invention (exemplary formulation G) in a dose of 200 mg Compound I (in either capsule, or tablet form) or as a comparative formulation (i.e. a suspension) to healthy human subjects under fasted conditions. See Example 3, infra, for details.

FIGS. 5 (A and B) are, respectively, graphs of the plasma concentration/time profiles of Compound I in eight individual healthy human subjects and the mean concentration/time profiles with error bars following once-a-day oral administration of 300 mg Compound I on a formulation of the present invention (exemplary formulation G) and 100 mg ritonavir for 10-days to the subjects under fed conditions. As a reference, the in vitro IC90 (28 ng/mL) of Compound I. See Example 3, infra for details.

FIG. 6 illustrates the in vitro dissolution profiles of two formulations of the present invention, each containing 100 mg of Compound 1.

FIG. 7 illustrates the in vitro dissolution profiles of two formulations of the present invention, i.e., Formulations U and V (see infra Table 3C).

### DETAILED DESCRIPTION OF THE INVENTION

### Definitions

Unless defined otherwise, all technical and scientific terms used herein have the same meaning as those commonly understood by one of ordinary skill in the art to which this invention belongs. Although methods and materials similar or equivalent to those described herein can be used in the practice or testing of the present invention, suitable methods and materials are described below. The materials, methods and examples are illustrative only, and are not intended to be limiting. All publications, patents and other documents mentioned herein are incorporated by reference in their entirety.

As used herein, the term “stable” with respect to an amorphous form of a compound refers to an amorphous form that is substantially free from crystalline form of the compound as assayed e.g., by X-ray diffraction. As used herein “substantially free” with respect to the amorphous form of Compound I as “substantially free” of crystalline form or solvate form means that the crystalline form or solvate form is present at <5% of total Compound I; preferably at ≦3% of total Compound I; more preferably at ≦2% of total Compound I.

As used herein, when administered “in combination” two (or more) therapeutic agents (e.g. Compound 1 and a cytochrome pH50 inhibitor) can be formulated as separate compositions which are administered at the same or different time(s), or the two (or more) therapeutic agents can be formulated in a combined fixed dosage form and administered as a single composition.

### Pharmaceutical Formulations

The present invention provides pharmaceutical formulations of Compound I in a solid molecular dispersion that meet the aforementioned need for enhanced bioavailability of Compound I. To prepare the formulations of the present invention, Compound I, in crystalline or amorphous form or a solvate of Compound I can be used as a starting material. Once the solid dispersions are formed, the formulations are substantially free of crystalline and solvate forms of Compound I. In the solid dispersions provided herein, Compound I in a stable amorphous form is uniformly dispersed in at least one suitable excipient, preferably a non-swellable polymer. The solid dispersions provided herein exhibit excellent mechanical and physical attributes necessary for milling, blending, and tablet compression. The solid dispersions of the present invention can be directly utilized as powders or granules. Alternatively, such solid dispersions can be used to prepare formulations in a variety of solid dosage forms including capsules, tablets, granules, powders, and unit dose packets. In fact, the solid dispersions provided herein are suitable for drug loading dosage forms with ≧100 mg drug per unit dosage form. The pharmaceutical formulations of the present invention provide an immediate release dissolution profile as well as sufficient bioavailability to reduce the number of doses administered per day to achieve the desired therapeutic plasma concentration(s) of Compound I.

Compound I has the following structure:

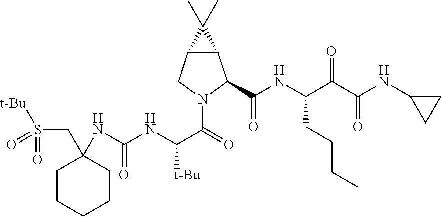

Compound I can be prepared according to International Patent Publication WO 2005/087731 (wherein Compound I is referred to as Compound 484) see, e.g., page 299, Example 792 to page 355, Example 833, which pages are specifically incorporated herein by reference.

Compound I is a neutral compound that exists in a crystalline or amorphous form. Compound I may also be converted to a crystalline solvate that is, a physical association of Compound I with one or more solvent molecules. The term “solvate” encompasses both solution-phase and isolatable solvates (e.g., when one or more solvent molecules are incorporated in the crystal lattice of the crystalline solid). Non-limiting examples of suitable solvates include ethanolates, methanolates, and the like. “Hydrate” is a solvate wherein the solvent molecule is H₂O. Preparation of solvates is generally known. A typical, non-limiting, process for preparing solvates involves dissolving a compound in desired amounts of the desired solvent (organic or water or mixtures thereof) at a higher than ambient temperature, and cooling the solution at a rate sufficient to form crystals which are then isolated by standard methods. Analytical techniques (e.g., I. R. spectroscopy. X-ray diffraction, etc.) show the presence of solvent in the crystals of a solvate.

The solid molecular dispersions and formulations of the present invention contain Compound I in amorphous form substantially free of crystalline and/or solvate forms.

Suitable polymers for use in the solid dispersions of the present invention include carbomer (i.e., a polymer of acrylic acid), hydroxypropyl cellulose, hydroxyethyl cellulose, hydroxypropylmethylcellulose, polyacrylate polymer, polyethylene oxide, polyvinyl alcohol, poloxamer, povidone, polytheylene glycol, copovidone, or a combination of two or more thereof. Polymers used as a solid dispersion agent may make up about 5% to about 95% by weight of the pharmaceutical formulation. In certain embodiments, polymer used as a solid dispersion agent is present at about 10% to about 90% by weight of the pharmaceutical formulation. In one preferred embodiment, polymer used as a solid dispersion agent is present at about 20% to about 80% by weight of the pharmaceutical formulation.

In certain preferred embodiments, the polymer is copovidone. Copovidone is commercially available, for example, from ISP or BASF. Copovidone is a copolymer of 1-vinyl-2-pyrrolidone and vinyl acetate in the mass proportion of 3:2.

In certain embodiments, Compound I in an amorphous form is stable within the solid dispersions disclosed herein after storage at 40° C. and 75% relative humidity for at least 3 months, preferably for at least 6 months.

In certain preferred embodiments, the ratio by weight of Compound I to polymer in the solid dispersion is in the range of about 10:1 to about 1:10. In certain other preferred embodiments, the ratio by weight of Compound I to polymer in the solid dispersion is in the range of about 1:1 to about 1:3. In one preferred embodiment, the ratio by weight of Compound I to polymer in the solid dispersion is about 1:1. In another preferred embodiment, the ratio by weight of Compound I to polymer in the solid dispersion is about 3:1.

In certain embodiments, the solid dispersions of the present invention may optionally further comprise one or more additional pharmaceutically acceptable excipients. In preferred embodiments, the solid dispersions of the present invention disclosed herein are formulated into pharmaceutical formulations in any of a variety of dosage forms for oral administration. Suitable pharmaceutical dosage forms include, but are not limited to, capsules, tablets, granules, powders, and unit dose packets. In one embodiment, the pharmaceutical formulation is enclosed in a capsule. In another embodiment, the pharmaceutical formulation is in the form of a tablet. In certain embodiments, dosage forms as described herein have a drug loading capacity of at least 100 mg, at least 200 mg, at least 300 mg, or at least 400 mg per oral unit dosage form.

Suitable pharmaceutically acceptable excipients are well known in the art. Exemplary diluents, surfactants, disintegrants, glidants, lubricants, and coating agents are provided below.

Examples of diluents include, without limitation, lactose, mannitol, xylitol, microcrystalline cellulose, calcium diphosphate, starch, calcium phosphate, sucrose, pregelatinized starch, calcium carbonate, calcium sulphate, powdered cellulose, microcrystalline cellulose (MCC, e.g., silicified MCC), cellulose acetate, compressible sugar, or a combination of two or more thereof. Diluents may make up about 5% to about 95% by weight of the pharmaceutical formulation. In certain embodiments, diluent is present at about 10% to about 90% by, weight of the pharmaceutical formulation. In one preferred embodiment, diluent is present at about 20% to about 80% by weight of the pharmaceutical formulation.

Examples of surfactants include, without limitation, hydrogenated vegetable oil, polyethylene sorbitan fatty acid ester, polyethylene stearate, polyoxyethylene alkyl ether, sorbitan ester (e.g., sorbitan fatty acid ester, Span), sodium lauryl sulfate, poloxamer; cremphor, capryol 90, docusate sodium, polyoxyehthylene castor oil derivative, triethyl citrate, or a combination of two or more thereof. Surfactants may make up about 0.2% to about 20% by weight of the pharmaceutical formulation. In certain embodiments, surfactant is present at about 0.5% to about 10% by weight of the pharmaceutical formulation. In one preferred embodiment, surfactant is present at about 2% to about 7% by weight of the pharmaceutical formulation.

Examples of disintegrants include, without limitation, starch, sodium starch glycolate, sodium alginate, calcium alginate; carboxymethyl cellulose sodium, carboxymethyl cellulose calcium, methyl cellulose, low-substituted hydroxypropylcellulose (L-HPC, e.g., LH-21, LH-B1), croscarmellose sodium, chitosan, crospovidone, guar gum, or a combination of two or more thereof. Disintegrants may make up about 0.5% to about 50% by weight of the pharmaceutical formulation. In certain embodiments, disintegrant is present at about 2% to about 20% by weight of the pharmaceutical formulation. In one preferred embodiment, disintegrant is present at about 5% to about 15% by weight of the pharmaceutical formulation.

Examples of glidants include, without limitation, sodium lauryl sulfate, silicon dioxide, calcium silicate, magnesium silicate, magnesium trisilicate, talc, or a combination of two or more thereof. Glidants may make up about 0.1% to about 10% by weight of the pharmaceutical formulation. In certain embodiments, glidant is present at about 0.2% to about 5% by weight of the pharmaceutical formulation. In one preferred embodiment, glidant is present at about 0.5% to about 3% by weight of the pharmaceutical formulation.

Examples of lubricants include, without limitation, stearic acid, magnesium stearate, calcium stearate, fat, wax, hydrogenated vegetable oil, castor oil, glycerin monostearate, glyceryl behenate, sodium stearyl fumurate, zinc stearate, glyceryl palmitostearate, medium-chain triglyceride, mineral oil, or a combination of two or more thereof. Lubricants may make up about 0.1% to about 10% by weight of the pharmaceutical formulation. In certain embodiments, lubricant is present at about 0.2% to about 5% by weight of the pharmaceutical formulation. In one preferred embodiment, lubricant is present at about 0.5% to about 3% by weight of the pharmaceutical formulation.

Examples of coating agents include, without limitation, carbomer (i.e., polymer of acrylic acid), cellulose acetate phthalate, hydroxypropyle cellulose, hydroxyethyl cellulose, hydroxypropyl methylcellulose, hydroxypropyl methylcellulose phthalate, polyacrylate polymer, polyvinyl alcohol, povidone, polytheylene glycol, copovidone, hypromellose acetate succinate, cellulose acetate, or a combination of two or more thereof. Coating agents may make up about 0.5% to about 20% by weight of the pharmaceutical formulation. In certain embodiments, coating agent is present at about 1% to about 15% by weight of the pharmaceutical formulation. In one preferred embodiment, coating agent is present at about 3% to about 7% by weight of the pharmaceutical formulation.

### Methods of Preparing Solid Dispersions

Another aspect of the invention provides methods of preparing the solid dispersions and formulations according to the present invention. The solid dispersions may be prepared by a hot melt extrusion process or preferably by a solvent evaporation process (e.g., spray drying).

In certain embodiments, solid dispersions of the present invention may be prepared using hot melt extrusion. According to the present invention, hot melt extrusion is used as a solvent-free, continuous process that melts one or more polymers and Compound I or a solvate thereof through an extruder with mechanical and thermal input. In certain embodiments, an optional plasticizer and/or an optional stabilizer is added to the mixture from which the solid dispersion is formed. In one embodiment, an acidifying ingredient (e.g., ascorbic acid) is added to the mixture from which solid dispersion is formed. In a embodiment, the mixture from which the solid dispersion is formed is blended prior to feeding into the extruder. In certain embodiments, a twin screw extruder is used whereby two screws concurrently turn to convey, mix, and melt the blend into a single homogenous solid dispersion.

The extrusion temperature is set such that both Compound I or a solvate thereof and polymer are completely melted and mixed through the extrusion process. Notably, the extrusion temperature and residence time of the mixture in the extruder are important factors affecting the level of degradation. The residence time being controlled by the feeding speed (i.e., the speed at which the material from which the solid dispersion is formed is fed into the extruder) and the rotation speed of the extruder's screw(s). In certain embodiments (e.g., exemplary formulations A-E detailed below), the extrusion temperature is between around 80° C. to around 95° C. and the feeding speed is in the range of between about 1.4 and about 1.5 lb/min with a screw rotation speed of between about 130 RPM and about 300 RPM.

In alternative preferred embodiments, solid dispersions of the present invention are prepared by dissolving both Compound I or a solvate thereof and polymer in an organic solvent followed by evaporation of the organic solvent. Dissolution of Compound I or a solvate thereof and polymer in the organic solvent may be accomplished at a temperature in the range of about 5° C. to about 70° C. Subsequent evaporation of the organic solvent is accomplished by heat, vacuum, spray drying, or a combination of two or more thereof. Suitable temperatures may be in the range of about 20° C. to about 80° C. Suitable organic solvents include, but are not limited to, ethanol, methanol, acetone, methylenechloride, dichloromethane, ethyl acetate, water, chloroform, toluene, or a combination of two or more thereof. In certain embodiments, a combination of organic solvents may be used, such as ethanol and acetone or methanol and acetone. Such combinations may be in any appropriate ratio in the range of 1:99 to 99:1 volume to volume. In certain preferred embodiments, the solid dispersions of the present invention are prepared by dissolving both Compound I or a solvate thereof and polymer in an organic solvent followed by evaporation of the organic solvent by spray drying at elevated temperature. In a preferred embodiment, Compound I and copovidine polymer (1:1) are dissolved in acetone.

Key process parameters for spray drying are the inlet N₂ temperature, outlet N₂ temperature, solution feed rate, percentage atomizing N₂ flow. Preferably, inlet N₂ temperature is between about 50° C. and about 90° C. and outlet N₂ temperature between about 25° C. and about 50° C. Preferably, the solution feed rate is between about 2.5 kg/h and about 3.5 kg/h. Preferably, the atomizing N₂ flow was between about 45% and about 55%.

In certain preferred embodiments, the ratio by weight of Compound I or a solvate thereof to polymer is in the range of about 10:1 to about 1:10. In certain embodiments, the ratio by weight of Compound I or a solvate thereof to polymer is in the range of about 2:1 to about 1:4, more preferably about 1:1 to about 1:3. In one embodiment, the ratio by weight of Compound I or a solvate thereof to polymer is about 1:1. In another embodiment, the ratio by weight of Compound I or a solvate thereof to polymer is about 1:3.

Pharmaceutical formulations of the present invention can be prepared using the following exemplary spray drying process.
Step 1: Dissolve Compound I or a solvate thereof and at least one polymer (e.g., copovidone) in organic solvent (e.g., acetone) to form a solution;
Step 2: Spray dry the solution prepared in Step 1 to obtain a spray dried solid dispersion;
Step 3: Dry the spray dried solid dispersion obtained from Step 2 in a suitable dryer to minimize residual organic solvent in the spray dried solid dispersion and obtain a dried solid dispersion;
Step 4: Blend the dried solid dispersion from Step 3 with one or more excipients (e.g., microcrystalline cellulose, lactose (e.g. lactose monohydrate), sodium lauryl sulfate, croscarmellose sodium) to form a blend;
Step 5: Mix the blend from Step 4 with lubricant (e.g., magnesium stearate) to form a lubricated blend;
Step 6: Roller compact the lubricated blend from Step 5 into a ribbon and mill the resultant ribbon into granules;
Step 7: Blend the granules from Step 6 with one or more additional excipients (e.g., colloidal silicone dioxide, sodium lauryl sulfate, croscarmellose sodium) to form a blend of granules;
Step 8: Mix lubricant (e.g., magnesium stearate) with the blend from Step 7.

For capsule dosage forms, the blend from step 8 is encapsulated. For tablet dosage forms, the blend from step 8 is compressed into core tablets. The tablet cores may optionally be film-coated, e.g., by spraying an aqueous dispersion of Opadry II White Y-30-18037 or Opadry II Yellow onto core tablets in a coater. In one embodiment, the film coating is in an amount that adds about 4% of the total weight of the uncoated tablet. In certain embodiments, the finished product is packaged into high density polyethylene (HDPE) bottles.

In certain alternative embodiments, the solid dispersion formed from steps 1 and/or 2 may be used directly as a pharmaceutical formulation. Thus, in these embodiments, each individual step subsequent to steps 1 and 2 is optional for formation of a pharmaceutical formulation. In certain embodiments, to improve granulation flow, the solid dispersion can be dry granulated using roller compaction and milling “as is” or after blending with one or more excipients. In certain other embodiments, the solid dispersion is processed without roller compaction and milling. In certain embodiments, the solid dispersion is blended with a lubricant to facilitate, high-throughput manufacture. Similarly, in certain embodiments, the solid dispersion is blended with a diluent to facilitate processing into suitable dosage forms.

Alternatively, pharmaceutical formulations of the present invention can be prepared using the following preferred exemplary spray drying process with fewer steps than described above herein.

Step A: Dissolve Compound 1 or a solvate thereof and at least one polymer (e.g. copovidone) in organic solvent (e.g. acetone) preferably in a 1:1 weight ratio to form a solution;

Step B: Spray dry the solution to obtain a spray dried solid dispersion;

Step C: Dry the solid dispersion obtained in Step B to obtain a dried dispersion;

Step D: Delump the dried dispersion;

Step E: Blend the dried dispersion with one or more excipients preferably delumped excipients(s) (e.g. microcrystalline cellulose, sodium lauryl sulfate, sodium croscarmellose (Ac-Di-Sol) and magnesium stearate) to form a blend;

Step F: compress the blend to form a tablet core and optimally,

Step G: coat the tablet core with a coating material (e.g. Opadry II).

As will be understood by those skilled in the art, delumping may be achieved by any known process including but not limited to co-milling.

For patient safety, the residual solvent (e.g., acetone) in solid dispersions prepared by the solvent evaporation process (e.g., spray drying) can be determined using a temperature programmed GC method. In brief, the analysis is performed using a DB-WAX, 0.25 μm film, 30 mm×0.32 mm ID column with helium as a carrier gas at a 1.3 mL/minute flow rate. Sample solutions are prepared by extracting a test sample in water:acetonitrile mixture, 10:90 v/v. For example, 400 mg of Compound I:copovidone (1:1) Spray-Dried Dispersion was extracted in water:acetonitrile mixture, 10:90 v/v; or 10 tablets of Compound I were extracted in water:acetonitrile mixture, 10:90 v/v. Standards are also prepared in water:acetonitrile mixture, 10:90 v/v. An external standard method is used to quantitate the organic solvent with flame ionization detection.

Methods of Treating or Ameliorating One or More Symptoms of HCV Infection or Disorders Associated with HCV Infection

Another aspect of the invention provides methods for treating or ameliorating one or more symptoms of HCV infection or disorders associated with HCV infection in a patient in need thereof comprising administering a pharmaceutical formulation of the present invention to the patient in need thereof. In preferred embodiments, the pharmaceutical formulations are administered in combination with a cytochrome P450 inhibitor. In certain preferred embodiments, the pharmaceutical formulations are administered in combination with a cytochrome P450 isoenzyme 3A4 (CYP3A4) inhibitor. In one preferred embodiment, the pharmaceutical formulations are administered in combination with ritonavir.

### Cytochrome P450 Inhibitors

In certain embodiments, at least one cytochrome P450 inhibitor is selected from the group of cytochrome P450 inhibitors referred to in the following documents (which are incorporated by reference herein):

WO2008049116, WO2008042240, WO2008022345, WO2007140299, WO2007111866, WO2007092616, WO2007071708, US20070149610, WO2007070834, WO2007034312, WO2007007060, WO2006108879, US20060222627, WO2006072881, WO2006024414, US20060009645, US20050171037, WO2005066162, WO2005042020, WO2005034963, US20050031713, US20040161479, WO2004060370, US20040047920, WO2003083052, US20010041706, WO2001058455, WO2000045817, WO9908676, WO9844939, WO9719112, WO9635415, US20080124407, WO2008027932, WO2008023273, WO2008013773, WO2008004100, WO2008004096, WO2007042037, WO2006136175, WO2006021456, WO2005007631, U.S. Pat. No. 6,686,338, U.S. Pat. No. 6,673,778, WO2002045704, WO2001087286, WO2000044933, WO9817667, WO2008023958, US20080045564, WO2008016709, U.S. Pat. No. 6,245,805, WO9715269, and WO9701349.

### CYP3A4 Inhibitors

In one embodiment, at least one CYP3A4 inhibitor is selected from the group of CYP3A4 inhibitors referred to in the following documents (which are incorporated by reference herein):

US20040052865A1, US20030150004A1, US20060099667A1, US20030096251A1, US20060073099A1, US20050272045A1, US20020061836A1, US20020016681A1, US20010041706A1, US20060009645A1, US20050222270A1, US20050031713A1, US20040254156A1, US20040214848A1, WO0173113A2, WO2005068611A1, US20050171037A1, WO2003089657A1, WO2003089656A1, WO2003042898A2, US20040243319A1, WO0045817A1, WO2006037993A2, WO2004021972A2, WO2006024414A2, WO2004060370A1, WO9948915A1, WO2006054755A1, WO2006037617A1, JP2006111597A, WO0111035A1, WO9844939A1, WO2003026573A2, WO2003047594A1, WO0245704A2, WO2005020962A1, WO2006021456A1, US20040047920A1, WO2003035074A1, WO2005007631A1, WO2005034963A1, WO2006061714A2, WO0158455A1, WO2003040121A1, WO2002094865A1, WO0044933A1, U.S. Pat. No. 6,673,778B1, WO2005098025A2, US20040106216A1, WO0017366A2, WO9905299A1, WO9719112A1, EP1158045A1, WO0034506A2, U.S. Pat. No. 5,886,157A, WO9841648A2, U.S. Pat. No. 6,200,754B1, U.S. Pat. No. 6,514,687B1, WO2005042020A2, WO9908676A1, WO9817667A1, WO0204660A2, WO2003046583A2, WO2003052123A1, WO2003046559A2, US20040101477A1, US20040084867A1, JP10204091A, WO9635415A2 WO9909976, WO98053658, US2004058982, U.S. Pat. No. 6,248,776, U.S. Pat. No. 6,063,809, U.S. Pat. No. 6,054,477, U.S. Pat. No. 6,162,479, WO2000054768, U.S. Pat. No. 6,309,687, U.S. Pat. No. 6,476,066, U.S. Pat. No. 6,660,766, WO 2004037827, U.S. Pat. No. 6,124,477, U.S. Pat. No. 5,820,915, U.S. Pat. No. 5,993,887, U.S. Pat. No. 5,990,154, U.S. Pat. No. 6,255,337, Fukuda et al., “Specific CYP3A4 inhibitors in grapefruit juice: furocoumarin dimers as components of drug interaction,” Pharmacogenetics, 7(5):391-396 (1997), Matsuda et al., “Taurine modulates induction of cytochrome P450 3A4 mRNA by rifampicin in the HepG2 cell line,” Biochim Biophys Acta, 1593(1):98-98 (2002); Tassaneeyakul et al., “Inhibition selectively of grapefruit juice components on human cytochromes P450,” Arch Biochem Biophys, 378(2):356-363 (2000); Widmer and Haun, “Variation in furanocoumarin content and new furanocoumarin dimmers in commercial grapefruit (Citrus paradise Macf.) juices,” Journal of Food Science, 70(4):C307-C312 (2005); and Arora et al., Drug Metab Dispos, 30(7):757-762 (2002).

Non-limiting examples of suitable CYP3A4 inhibitors include ketoconazole (Nizoral™, commercially available from Janssen Pharmaceutica), itraconazole (Sporanox®, commercially available from Janssen-Cilag), ritonavir (Norvir® commercially available from Abbott), nelfinavir (Viracept® commercially available from Pfizer), indinavir (Crixivan® commercially available from Merck & Co., Inc), erythromycin (Akne-Mycin®, A/T/S®, Emgel®, Erycette®, EryDerm®, Erygel®, Erymax®, Ery-Sol®, Erythra-Derm®, ETS®, Staticin®, Theramycin Z®, T-Stat®, ERYC®, Ery-Tab®, Erythromycin Base Filmtab®, PCE® Dispertab®), clarithromycin (Biaxin®), troleandomycin (Tao®), saquinavir, nefazodone, fluconazole, grapefruit juice, fluoxetine (Prozac® commercially available from Eli Lilly and Company, Zoloft® commercially available from Pfizer Pharmaceuticals, Anafranil® commercially available from Mallinckrodt Inc.), fluvoxamine (Luvox®), Zyflo (Zileuton® commercially available from Abbott Laboratories), clotrimazole (Fungoid® Solution, Gyne-Lotrimin®, GyneLotrimin® 3, Gyne-Lotrimin® 3 Combination Pack, Gyne-Lotrimin®-3, Lotrim® AF Jock Itch Cream, Lotrimin®, Lotrimin® AF, Mycelex® Troche, Mycelex®-7), midazolam (available from Apotex Corp.), naringenin, bergamottin, BAS 100 (available from Bioavailability Systems). In one preferred embodiment, the CYP3A4 inhibitor is ketoconazole (Nizoral™) or clarithromycin (Biaxin®). In another preferred embodiment, the CYP3A4 inhibitor is BAS 100 (available from Bioavailability Systems). In yet another preferred embodiment, the CYP3A4 inhibitor is AVI-4557.

AVI-4557, also known as NeuGene® (available from AVI Biopharma, Inc.) is an antisense phosphorodiamidate morpholino oligomer (PMO) that inhibits targeted gene expression by preventing ribosomal assembly, thus preventing translation. Specifically, AVI-4557 is a 20-mer PMO with the sequence 5′-CTGGGATGAGAGCCATCACT-3′ that inhibits CYP3A4. AVI-4557 can be absorbed when given orally. In certain preferred embodiments, AVI-4557 is administered orally at a dosage of about 10 mg per day. Alternatively, AVI-4557 may be administered intravenously or subcutaneously.

Preferably, the clarithromycin is administered at a unit dosage sufficient to increase the bioavailability of the HCV protease inhibitor. Preferably, the clarithromycin is administered at a unit dosage of about 5 mg to about 249 mg per day. Preferably, the clarithromycin is administered at a unit dosage of 5 mg, 10 mg, 15 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, or 249 mg per day.

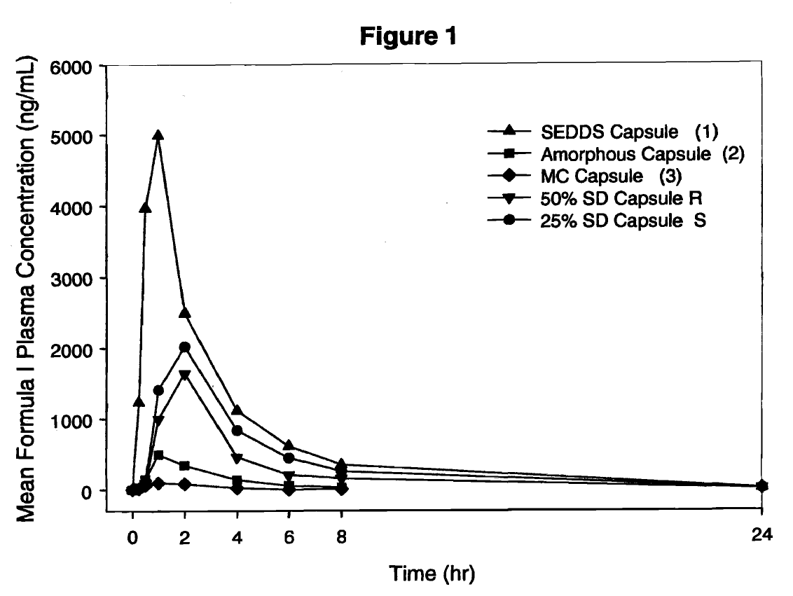

In addition, non-limiting examples of suitable compounds that inhibit HIV protease which have also been identified as CYP3A4 inhibitors are disclosed in US 2005/0209301 (at page 3, paragraph [0025] to page 5, paragraph [0071] and page 10, paragraph [0170] to page 12, paragraph [0226]) as well as US 2005/0267074 (at page 3, paragraph [0025], paragraph to page 7, paragraph [0114], page 7, paragraph [0119] to paragraph [0124], and FIG. 1-3) incorporated herein by reference. The following is a list of specific compounds depicted in US 2005/0209301: {1-Benzyl-3-[(3-dimethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dimethylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; [1-Benzyl-3-({3-[(ethyl-methyl-amino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(ethyl-methyl-amino)-ethylidene]-2-oxo-2,3-dihyd-ro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(methyl-propyl-amino)-methylene-]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[1-(methyl-propyl-amino)-ethylid-ene]-2-oxo-2,3-dihydro-1H-indole-5-sulfonyl}-amino)-propyl]-carbamic acid hexahydro-furo[2,3-b]furan-3-yl ester; {1-Benzyl-3-[(3-diethylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-diethylamino-ethylidene)-2-oxo-2,3-dihydro-1H-in-dole-5-sulfonyl]-isobutyl-amino}-2-hydroxy-propyl)-carbamic acid hexahydro-furo[2,3-b]furan-3-yl ester; {1-Benzyl-3-[(3-dipropylaminomethylene-2-oxo-2,3-dihydro-1H-indole-5-sulfonyl)-isobutyl-amino]-2-hydroxy-propyl}-carbamic acid hexahydro-furo[2,3-b]furan-3-yl ester; (1-Benzyl-3-{[3-(1-dipropylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; {1-Benzyl-2-hydroxy-3-[isobutyl-(2-oxo-3-piperidin-1-ylmethylene-2,-3-dihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(1-piperidin-1-yl-ethylide-ne)-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-piperazin-1-ylmethylene-2,-3-dihydro-1H-indole-5-sulfonyl)-amino]-propyl}-carbamic acid hexahydro-furo[2,3-b]furan-3-yl ester; {1-Benzyl-2-hydroxy-3-[isobutyl-(3-morpholin-4-ylmethylene-2-oxo-2,-3-dihydro-1H-indole-5-sulfonyl)-amino]-propyl}-carbamic acid hexahydro-furo[2,3-b]furan-3-yl ester; {3-[(3-Aminomethylene-2-oxo-2,3-dihydro-1H-indole-5-sulfonyl)-isobu-tyl-amino]-1-benzyl-2-hydroxy-propyl}-carbamic acid hexahydro-furo[2,3-b]furan-3-yl ester; (3-{[3-(1-Amino-ethylidene)-2-oxo-2,3-dihydro-1H-indole-5-sulfonyl]-1-isobutyl-amino}-1-benzyl-2-hydroxy-propyl)-carbamic acid hexahydro-furo[2,3-b]furan-3-yl ester; {1-Benzyl-2-hydroxy-3-[isobutyl-(3-methylaminomethylene-2-oxo-2,3-d-ihydro-1H-indole-5-sulfonyl)-amino]-propyl}-carbamic acid hexahydro-furo[2,3-b]furan-3-yl ester; (1-Benzyl-2-hydroxy-3-{isobutyl-[3-(1-methylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-3-[(3-ethylaminomethylene-2-oxo-2,3-dihydro-1H-indole-5-s-ulfonyl)-isobutyl-amino]-2-hydroxy-propyl}-carbamic acid hexahydro-furo[2,3-b]furan-3-yl ester; (1-Benzyl-3-{[3-(1-ethylamino-ethylidene)-2-oxo-2,3-dihydro-1H-indo-le-5-sulfonyl]-isobutyl-amino}-2-hydroxy-propyl)-carbamic acid hexahydro-furo[2,3-b]furan-3-yl ester; [1-Benzyl-2-hydroxy-3-(isobutyl-{2-oxo-3-[(2,2,2-trifluoro-ethylami-no)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[1-(2,2,2-trifluoro-ethyla-mino)-ethylidene]-2,3-dihydro-1H-indole-5-sulfonyl}-amino)-propyl]-carbamic acid hexahydro-furo[2,3-b]furan-3-yl ester; [1-Benzyl-2-hydroxy-3-({8 3-[(2-hydroxy-ethylamino)-methylene]-2-oxo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-({3-[1-(2-hydroxy-ethylamino)-ethylidene]-2-o-xo-2,3-dihydro-1H-indole-5-sulfonyl}-isobutyl-amino)-propyl]-carbamic acid hexahydro-furo[2,3-b]furan-3-yl ester; [1-Benzyl-2-hydroxy-3-(isobutyl-{3-[(2-methoxy-ethylamino)-methylen-e]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[1-(2-methoxy-ethylamino)-ethyli-dene]-2-oxo-2,3-dihydro-1H-indole-5-sulfonyl}-amino)-propyl]-carbamic acid hexahydro-furo[2,3-b]furan-3-yl ester; [1-Benzyl-3-({3-[(2-dimethylamino-ethylamino)-methylene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(2-dimethylamino-ethylamino)-ethylidene]-2-oxo-2-,3-dihydro-1H-indole-5-sulfonyl}-isobutyl-amino)-2-hydroxy-propyl]-carbami-c acid hexahydro-furo[2,3-b]furan-3-yl ester; (1-Benzyl-2-hydroxy-3-{isobutyl-[3-(isopropylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isopropylamino-ethylidene)-2-oxo-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-propylaminomethylene-2,3-d-ihydro-1H-indole-5-sulfonyl)-amino]-propyl}-carbamic acid hexahydro-furo[2,3-b]furan-3-yl ester; (1-Benzyl-2-hydroxy-3-{isobutyl-[2-oxo-3-(1-propylamino-ethylidene)-2,3-dihydro-1H-indole-5-sulfonyl]-amino}-propyl)-carbamic acid hexahydro-furo[2,3-b]furan-3-yl ester; {1-Benzyl-2-hydroxy-3-[isobutyl-(2-oxo-3-pyrrolidin-2-ylidene-2,3-d-ihydro-1H-indole-5-sulfonyl)-amino]-propyl}-carbamic acid hexahydro-furo[2,3-b]furan-3-yl ester; {1-Benzyl-3-[(3-butylaminomethylene-2-oxo-2,3-dihydro-1H-indole-5-s-ulfonyl)-isobutyl-amino]-2-hydroxy-propyl}-carbamic acid hexahydro-furo[2,3-b]furan-3-yl ester; (1-Benzyl-3-{[3-(1-butylamino-ethylidene)-2-oxo-2,3-dihydro-1H-indo-le-5-sulfonyl]-isobutyl-amino}-2-hydroxy-propyl)-carbamic acid hexahydro-furo[2,3-b]furan-3-yl ester; (1-Benzyl-2-hydroxy-3-{isobutyl-[3-(isobutylamino-methylene)-2-oxo-2,3-dihydro-1H-indole-5-sulfonyl]-amino}-propyl)-carbamic acid hexahydro-furo[2,3-b]furan-3-yl ester; (1-Benzyl-2-hydroxy-3-{isobutyl-[3-(1-isobutylamino-ethylidene)-2-o-xo-2,3-dihydro-1H-indole-5-sulfonyl]-amino}-propyl)-carbamic acid hexahydro-furo[2,3-b]furan-3-yl ester; (1-Benzyl-3-{[3-(tert-butylamino-methylene)-2-oxo-2,3-dihydro-1H-in-dole-5-sulfonyl]-isobutyl-amino}-2-hydroxy-propyl)-carbamic acid hexahydro-furo[2,3-b]furan-3-yl ester; (1-Benzyl-3-{[3-(1-tert-butylamino-ethylidene)-2-oxo-2,3-dihydro-1H-indole-5-sulfonyl]-isobutyl-amino}-2-hydroxy-propyl)-carbamic acid hexahydro-furo[2,3-b]furan-3-yl ester; [1-Benzyl-3-({3-[(2,2-dimethyl-propylamino)-methylene]-2-oxo-2,3-di-hydro-1H-indole-5-sulfonyl}-isobutyl-amino)-2-hydroxy-propyl]-carbamic acid hexahydro-furo[2,3-b]furan-3-yl ester; [1-Benzyl-3-({3-[1-(2,2-dimethyl-propylamino)-ethylidene]-2-oxo-2,3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(2-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{3-[(3-methyl-butylamino)-methylene-]-2-oxo-2,3-dihydro-1H-indole-5-sulfonyl}-amino)-propyl]-carbamic acid hexahydro-furo[2,3-b]furan-3-yl ester; [1-Benzyl-3-({3-[(3,3-dimethyl-butylamino)-methylene]-2-oxo-2,3-dih-ydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{3-[(1-isopropyl-2-methyl-propylami-no)-methylene]-2-oxo-2,3-dihydro-1H-indole-5-sulfonyl}-amino)-propyl]-carb-amic acid hexahydro-furo[2,3-b]furan-3-yl ester; {1-Benzyl-2-hydroxy-3-[isobutyl-(2-oxo-3-phenylaminomethylene-2,3-d-ihydro-1H-indole-5-sulfonyl)-amino]-propyl}-carbamic acid hexahydro-furo[2,3-b]furan-3-yl ester; (1-Benzyl-3-{([3-(benzylamino-methylene)-2-oxo-2,3-dihydro-1H-indol-e-5-sulfonyl]-isobutyl-amino}-2-hydroxy-propyl)-carbamic acid hexahydro-furo[2,3-b]furan-3-yl ester; (1-Benzyl-3-{[3-(1-benzylamino-ethylidene)-2-oxo-2,3-dihydro-1H-ind-ole-5-sulfonyl]-isobutyl-amino}-2-hydroxy-propyl)-carbamic acid hexahydro-furo[2,3-b]furan-3-yl ester; [1-Benzyl-3-({3-[(cyclohexylmethyl-amino)-methylene]-2-oxo-2,3-dihy-dro-1H-indole-5-sulfonyl}-isobutyl-amino)-2-hydroxy-propyl]-carbamic acid hexahydro-furo[2,3-b]furan-3-yl ester; {1-Benzyl-2-hydroxy-3-[isobutyl-(2-oxo-3-{[(pyridin-4-ylmethyl)-ami-no]-methylene}-2,3-dihydro-1H-indole-5-sulfonyl)-amino]-propyl}-carbamic acid hexahydro-furo[2,3-b]furan-3yl ester; (1-Benzyl-2-hydroxy-3-{isobutyl-[2-oxo-3-(phenethylamino-methylene)-2,3-dihydro-1H-indole-5-sulfonyl]-amino}-propyl)-carbamic acid hexahydro-furo[2,3-b]furan-3-yl ester; [1-Benzyl-3-({3-[(2-cyclohex-1-enyl-ethylamino)-methylene]-2-oxo-2,-3-dihydro-1H-indole-5-sulfonyl}-isobutyl-amino)-2-hydroxy-propyl]-carbamic acid hexahydro-furo[2,3-b]furan-3-yl ester; [1-Benzyl-2-hydroxy-3-(isobutyl-{2-oxo-3-[(2-pyridin-2-yl-ethylamin-o)-methylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(2-phenyl-propylamino)-me-thylene]-2,3-dihydro-1H-indole-5-sulfonyl}-amino)-propyl]-carbamic acid hexahydro-furo[2,3-b]furan-3-yl ester; [1-Benzyl-2-hydroxy-3-(isobutyl-{2-oxo-3-[(4-phenyl-butylamino)-met-hylene]-2,3-dihydro-1H-indole-5-sulfonyl}-amino)-propyl]-carbamic acid hexahydro-furo[2,3-b]furan-3-yl ester; {1-Benzyl-2-hydroxy-3-[isobutyl-(3-nonylaminomethylene-2-oxo-2,3-di-hydro-1H-indole-5-sulfonyl)-amino]-propyl}-carbamic acid hexahydro-furo[2,3-b]furan-3-yl ester; and (1-Benzyl-2-hydroxy-3-{[3-(1-hydroxy-ethylidene)-2-oxo-2,3-dihydro-1H-indole-5-sulfonyl]-isobutyl-amino}-propyl)-carbamic acid hexahydro-furo[2,3-b]furan-3-yl ester; and the pharmaceutically acceptable salts thereof, as single stereoisomers or mixtures of stereoisomers. Notably, US 2005/0267074 emphasizes that compounds having a benzofuran moiety are potent inhibitors of CYP3A4. HIV inhibitors useful as CYP3A4 inhibitors are also disclosed in U.S. Patent Publication No. US 20070287664, incorporated herein by reference.

In one embodiment, at least one CYP3A4 inhibitor is selected from the compounds disclosed in one or more of the following patent applications assigned to Sequoia Pharmaceuticals, Inc., the disclosure of each of which is incorporated herein by reference: U.S. Patent Publication No. US 2005/0209301 and U.S. Patent Publication No. US 2005/0267074.

In one embodiment, at least one CYP3A4 inhibitor is selected from the compounds disclosed in one or more of the following patents and patent applications assigned to Bioavailability Systems, LLC, the disclosure of each of which is incorporated herein by reference: US 2004058982, U.S. Pat. No. 6,248,776, U.S. Pat. No. 6,063,809, U.S. Pat. No. 6,054,477, U.S. Pat. No. 6,162,479, WO 2000054768, U.S. Pat. No. 6,309,687, U.S. Pat. No. 6,476,066, U.S. Pat. No. 6,660,766, WO 2004037827, U.S. Pat. No. 6,124,477, U.S. Pat. No. 5,820,915, U.S. Pat. No. 5,993,887, U.S. Pat. No. 5,990,154, U.S. Pat. No. 6,255,337. In particular, see, U.S. Pat. No. 6,063,809, column 5, line 30 to column 12, line 65; WO 2000054768, page 10, line 11 to page 22, line 1, and WO 2004037827, page 4 to page 17, incorporated herein by reference.

According to certain preferred embodiments of the present invention, at least one CYP3A4 inhibitor is ritonavir, ketoconazole, clarithromycin, BAS100, a compound disclosed in U.S. Patent Publication No. US 2005/0209301 or U.S. Patent Publication No. US 2005/0267074, a pharmaceutically acceptable salt, solvate or ester thereof, or AVI-4557. In one embodiment, at least one CYP3A4 inhibitor is ritonavir or a pharmaceutically acceptable salt, solvate or ester thereof. In another embodiment, at least one CYP3A4 inhibitor is ketoconazole or a pharmaceutically acceptable salt, solvate or ester thereof. In another embodiment, at least one CYP3A4 inhibitor is clarithromycin or a pharmaceutically acceptable salt, solvate or ester thereof. In another embodiment, at least one CYP3A4 inhibitor is a compound disclosed in U.S. Patent Publication No. US 2005/0209301 or U.S. Patent Publication No. US 2005/0267074 or a pharmaceutically acceptable salt, solvate or ester thereof. In one embodiment, at least one CYP3A4 inhibitor is AVI-4557. In another embodiment, at least one CYP3A4 inhibitor is BAS 100 or a pharmaceutically acceptable salt, solvate or ester thereof. Notably, at least one CYP3A4 inhibitor is identified by the Chemical Abstracts Services (CAS) Number 684217-04-7 which corresponds to the Chemical Abstract index name 7H-Furo[3,2-g][1]benzopyran-7-one, 4-[[(2E)-5-[(4R)-4′-[[(2E)-3,7-dimethyl-2,6-octadienyl]oxy]-5,5-dimethylspiro[1,3-dioxolane-2,7′-[7H]furo[3,2-g][1]benzopyran]-4-yl]-3-methyl-2-pentenyl]oxy]; the CAS Number 684217-03-6 which corresponds to the Chemical Abstract index name 7H-Furo[3,2-g][1]benzopyran-7-one, 4-[[(2E)-5-[(4R)-4′-[[2E)-6,7-dihydroxy-3,7-dimethyl-2-octenyl]oxy]-5,5-dimethylspiro[1,3-dioxolane-2,7′-[7H]furo[3,2-g][1]benzopyran]-4-yl]-3-methyl-2-pentenyl]oxy], or the CAS Number 267428-36-4 which corresponds to the Chemical Abstract index name 7H-Furo[3,2-g][1]benzopyran-7-one, 4-[[(2E)-5-[(2R,4R)-4′-[[(2E,6R)-6,7-dihydroxy-3,7-dimethyl-2-octenyl]oxy]-5,5-dimethylspiro[1,3-dioxolane-2,7′[7H]furo[3,2-g][1]benzopyran]-4-yl]-3-methyl-2-pentenyl]oxy]; all of which is further described in WO 2004037827. In one embodiment, at least one CYP3A4 inhibitor has the structure shown below:

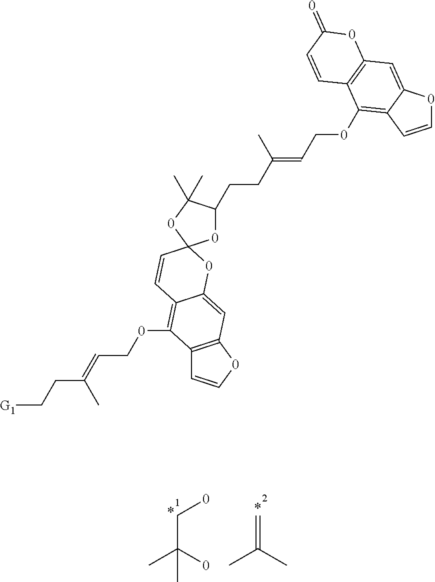

An effective amount of CYP3A4 inhibitor is an amount effective to increase the bioavailability of Compound I, an HCV protease inhibitor. For any CYP3A4 inhibitor, the effective amount can be estimated initially either in cell culture assays or in a relevant animal model, such as monkey. The animal model may also be used to determine the appropriate concentration range and route of administration. Such information can be then be used to determine useful doses and routes for administration in humans.

The amount and frequency of administration of Compound I or a pharmaceutically acceptable salt thereof will be regulated according to the judgment of the attending clinician considering such factors as age, condition, size of the patient as well as severity of the symptoms being treated. In most preferred embodiments, the pharmaceutical formulations comprising Compound I and polymer described herein are administered to a patient in need thereof thrice-a-day (TID), twice-a-day (BID), or once-a-day (QD). In one embodiment, the pharmaceutical formulations comprising Compound I and polymer described herein are administered to a patient in need thereof every 8 hours, every 12 hours, or every 24 hours. A typical recommended daily dosage regimen for treating or ameliorating one or more symptoms of HCV or disorders associated with HCV in a subject can range from about 100 mg/day to about 4800 mg/day Compound I. In certain preferred embodiments, the recommended daily dosage regimen for treating or ameliorating one or more symptoms of HCV or disorders associated with HCV in a subject can range from about 600 mg TID to about 1600 mg TID Compound I. Such TID dosage regimens can be administered in the absence of a cytochrome P450 inhibitor. In other embodiments, the pharmaceutical formulations of the present invention are administered in combination with a cytochrome P450 inhibitor, preferably a CYP3A4 inhibitor (e.g., ritonavir, preferably at a dose of 100 mg ritonavir administered either QD or BID).

The recommended daily dosage regimen for treating or ameliorating one or more symptoms of HCV or disorders associated with HCV in a subject can range from about 100 mg BID to about 400 mg BID Compound I in combination with a cytochrome P450 inhibitor (e.g., about 100 mg ritonavir BID). In yet other embodiments, the recommended daily dosage regimen for treating or ameliorating one or more symptoms of HCV or disorders associated with HCV in a subject can range from about 100 mg QD to about 600 mg QD Compound I in combination with a cytochrome P450 inhibitor (e.g., about 100 mg ritonavir QD).

In certain such embodiments, a dose comprises at least one oral dosage form. In certain such embodiments, a dose may comprise at least one additional oral dosage form administered simultaneously with the first dosage form, or within about 5 minutes, or even ten minutes of the first oral dosage form.

The pharmaceutical formulations of the present invention are administered to a patient according to a dosing regimen. It should be understood that the specific dosing regimen for any particular patient will depend on a variety of factors, including species, age, body weight, body surface area, height, general health, sex, diet, time of administration, rate of excretion, drug combination, specific disease being treated, the severity of the condition, the renal and hepatic function of the patient, the particular active ingredient employed, and the judgment of the treating physician.

Other features and embodiments of the invention will become apparent by the following examples which are given for illustration of the invention rather than limiting its intended scope.

### EXAMPLES

### Example 1

### Preparation of Pharmaceutical Formulations

Exemplary solid molecular dispersions of the present invention prepared by hot melt extrusion are detailed in Table 1A.

TABLE 1A
Exemplary solid dispersions A-E prepared by hot melt extrusion

| | Formulation | | | | |
|---|---|---|---|---|---|
| Ingredients (mg) | A | B | C | D | E |
| Compound I or a | 150 | 30 | 150 | 30 | 30 |
| solvate thereof | | | | | |
| Copovidone | 150 | 30 | 150 | 30 | 30 |
| Triethyl Citrate | 15 | 3 | — | — | — |
| Vitamin E TPGS¹ | — | — | — | 1.5 | — |
| Span 20² | — | — | — | — | 1.5 |
| Lactic Acid | — | — | 15 | 1.5 | — |
| Stearic Acid | — | — | — | — | 1.5 |
| Succinic Acid | — | 1.5 | — | — | — |

¹Vitamine E, d, α-Tocophenyl polyethylene glycol 1000 succinate available from Eastman Chem. Co,; Kingsport, TN.
²Sorbitan laurate, a/k/a sorbitan mono dodecanoate, available from Sigma Aldrich, St. Louis, MO.

Likewise, exemplary pharmaceutical formulation F was prepared using hot melt extrusion to form a solid dispersion (as in exemplary solid dispersion A wherein Compound I, Copovidone, and triethyl citrate are present in a ratio by weight of 1:1:0.1) which was subsequently blended with the remaining excipients detailed in Table 1B. The final blend was either encapsulated for a capsule dosage form or compressed to form a tablet core.

TABLE 1B
Exemplary pharmaceutical formulation F

| Ingredients (mg) | Formulation F |
|---|---|
| Compound I or a | 400 |
| solvate thereof | |
| Copovidone | 400 |
| Triethyl Citrate | 40 |
| Sodium Lauryl Sulfate | 40 |
| Sodium Croscarmellose | 340 |
| Pregelatinized Starch | 100 |
| Silicon Dioxide | 2.2 |
| Magnesium Stearate | 2.2 |

The solid dispersions described in Tables 1A and Table 1B were prepared using Compound I and the polymer copovidone as well as an optional plasticizer and/or optional stabilizer under the hot melt extrusion conditions described in Table 2.

TABLE 2
Hot melt extrusion conditions

| | Zone | | |
|---|---|---|---|
| | Temperature | Rotation Speed | Feed |
| Sample | (° C.) | (rpm) | (lb/min) |
| A | 90 | 250-300 | 1.4 |
| B | 90 | 300 | 1.4 |
| C | 90-95 | 130-200 | 1.5 |
| D | 90 | 150 | 1.4 |
| E | 80-90 | 230 | 1.5 |

Additional exemplary pharmaceutical formulations of the present invention are detailed in Tables 3A and 3B.

TABLE 3A
Exemplary pharmaceutical formulations G-Q

| Ingredients | Formulation | | | | | | | | | | |
|---|---|---|---|---|---|---|---|---|---|---|---|
| (mg) | G | H | I | J | K | L | M | N | O | P | Q |
| Compound I or | 100 | 100 | 100 | 100 | 100 | 100 | 100 | 100 | 100 | 100 | 100 |
| a solvate | | | | | | | | | | | |
| thereof | | | | | | | | | | | |
| Copovidone | 100 | 100 | 100 | 100 | 100 | 100 | 100 | 100 | 100 | 73.9 | 135.3 |
| MCC, Avicel | 50 | 72.2 | 45.8 | 54.2 | 65.6 | 34.4 | 59.6 | 40.4 | 27.8 | 76.1 | 14.7 |
| PH 102 | | | | | | | | | | | |
| Lactose Mono. | 86 | 73.1 | 98.9 | 73.1 | 73.1 | 98.9 | 73.1 | 98.9 | 98.9 | 86 | 86 |
| Spray Dried | | | | | | | | | | | |
| Sodium Lauryl | 20 | 17 | 17 | 23 | 23 | 17 | 17 | 23 | 23 | 20 | 20 |
| Sulfate | | | | | | | | | | | |
| Sodium | 40 | 34 | 34 | 46 | 34 | 46 | 46 | 34 | 46 | 40 | 40 |
| Croscarmellose | | | | | | | | | | | |
| Magnesium | 2 | 1.7 | 2.3 | 1.7 | 2.3 | 1.7 | 2.3 | 1.7 | 2.3 | 2 | 2 |
| Stearate | | | | | | | | | | | |
| Colloidal | 2 | 2 | 2 | 2 | 2 | 2 | 2 | 2 | 2 | 2 | 2 |
| Silicon Dioxide | | | | | | | | | | | |

TABLE 3B
Exemplary pharmaceutical formulations R-T

| | Formulation | | |
|---|---|---|---|
| Ingredients (mg) | R | S | T |
| Compound I or a | 100 | 50 | 133 |
| solvate thereof | | | |
| Copovidone | 100 | 150 | 133 |
| Crospovidone | 10 | 10 | — |
| Sodium Croscarmellose | — | — | 80 |
| Microcrystalline Cellulose | — | — | 33.3 |
| Sodium Lauryl Sulfate | 10 | 5 | 13.3 |
| Silicon Dioxide | 1.25 | 1.25 | 0.73 |
| Magnesium Stearate | 0.63 | 0.63 | 0.73 |
| Pregelatinized Starch | 50 | 25 | 33.3 |

Solid dispersions of Compound I and copovidone used to prepare exemplary Formulations G-T according to the present invention were prepared using solvent evaporation (specifically, spray drying) as described in the section above entitled “Methods of Preparing Solid Dispersions” following Steps 1-8 of the exemplary spray drying process. The solid dispersions were subsequently blended with the remaining excipients detailed in Tables 3A and 3B. The final blend was either encapsulated for a capsule dosage form or compressed to form a tablet core.

Solid dispersions of Compound I and Copovidone (commercially available as Plasdone S630™ from ISP, USA) were prepared using solvent evaporation as described in the section above entitled “Methods of Preparing Solid Dispersion” following Steps A-G of the exemplary spray drying process using the ingredients set forth in Table 3C below.

TABLE 3C
Exemplary Pharmaceutical Formulations U - V

Formulations

| Ingredients (mg) | U (mg/tablet) | V (mg/tablet) |
|---|---|---|
| Compound I or solvate | 100 | 300 |
| Copovidone (Plasdone S- | 100 | 300 |
| 630) | | |
| Microcrystalline Cellulose | 52 | 156 |
| (PH 102) | | |
| Sodium Lauryl Sulfate | 15 | 45 |
| Sodium Croscarmellose | 30 | 90 |
| (Ac-Di-Sol) | | |
| Colloidal Silicon Dioxide | 1.5 | 4.5 |
| (Cab-O-Sil) | | |
| Magnesium Stearate | 1.5 | 4.5 |
| Film Coat Opadry II Yellow | 12.0 | 36.0 |

### Stability of Solid Dispersion Bulk Powder and Pharmaceutical Formulations

Solid dispersions were assessed for crystallinity e.g., by X-ray diffraction (XRD) as detailed below. In brief, XRD was carried out as follows. A sample was prepared on a zero-background shallow cavity X-ray specimen holder and analyzed using a Rigaku D/Max 2200 diffractometer. The diffractometer was configured in Bragg-Brentano geometry and equipped with theta-compensating divergence and anti-scatter slits and a 0.2 mm fixed receive slit. Monochromatization was achieved using a diffracted beam graphite monochromator. The detector used was a scintillation counter with pulse height analyzer. The sample was scanned from 5-30° 2-theta with a step size of 0.02° and a scan rate of at least 5 seconds per step. The collected diffraction patterns were visually observed for the presence of discrete diffraction peaks indicating the presence of crystallinity. Using the aforementioned XRD technique, the lower detection limit for crystalline Compound I was 2%.

XRD analyses of the solid dispersions detailed in Table 1A prepared by hot melt extrusion confirmed the absence of crystallinity in all samples tested. Likewise, no crystallinity was detected by XRD in any of the solid molecular dispersions of Compound I and copovidone formed in the ratios detailed in Table 3A and 3B which were prepared by spray drying. Furthermore, no crystallinity was detected in samples of solid dispersion bulk powder prepared by spray drying that were stored for 6 months under various conditions (i.e., 5° C./ambient RH in a closed bottle, 25° C./60% relative humidity (RH)) in either an open or closed bottle, 30° C./ambient RH in a closed bottle, 40° C./75% RH in a closed bottle, or 50° C./ambient RH in a closed bottle). These results indicate that Compound I in an amorphous form is stable within the solid molecular dispersions of the present invention for a commercially acceptable shelf-life of at least 1 year.

Solid dispersion bulk powder as well as pharmaceutical formulations formed therefrom were also assessed at various timepoints (up to 6 months) under ambient or accelerated conditions for moisture content, label strength of Compound I, and the presence of Compound I degradation products.

The moisture content in solid dispersion bulk powder and tablets was assessed using a Karl Fischer titrator. A test sample was prepared in formamide:methanol (2:1, v:v) (e.g., from either (i) a single weighing of 500 mg solid dispersion bulk powder, or (ii) a single composite of 10 tablets), then sonicated, rotated, and centrifuged. The test sample solution was titrated using the coulometric Karl Fischer titrator and the moisture content (water) was reported in percent.

Content uniformity and identification of Compound I in tablets was assessed using reverse-phase HPLC. The analysis was performed using an XBridge 3.5 μC18, 4.6×50 mm column maintained at 30° C. Isocratic elution was used with a mobile phase consisting of 0.05% ammonium hydroxide:methanol (30:70 v/v) with a 1.5 ml/minute flow rate. An external standard method of quantitation was used with UV detection at 220 nm. Sample and standard solutions were prepared in an acidified methanol diluent to contain 1 mg/mL Compound I. Identification of Compound I in tablets was verified by the HPLC retention time ratio of Compound I in the sample and reference standard solutions.

Similarly, the presence of degradation products of Compound I in solid dispersion bulk powder and tablets was assessed using reverse-phase high performance liquid chromatography (HPLC). In brief, HPLC analysis was performed using an ACE 3 C18 (150×4.6 mm, 3 μm) column maintained at 20° C. with gradient elution. Mobile phase A contained methanol: acetonitrile: 20 mM phosphate buffer pH 7.0 (50:17:33, v/v/v). Mobile phase B contained methanol: acetonitrile: 20 mM phosphate buffer pH 7.0 (55:35:10, v/v/v). Ultraviolet (UV) detection was used at 220 nm. For the determination of Compound I degradation products in bulk powder or tablets as well as the identification of Compound I in bulk powder, a 5 mg/mL sample solution was prepared in extraction solvent (0.01% trifluoroacetic acid in methanol) for analysis. External standard solutions were prepared for the quantitation of Compound I. Identification of Compound I in sample solutions was verified by the HPLC retention time ratio of Compound I in the sample and reference standard solutions.

Solid dispersion bulk powder prepared by hot melt extrusion had significant levels of the inactive epimer of Compound I as detected by HPLC. Even though the level of epimerization was relatively lower in solid dispersion prepared by hot melt extrusion using amorphous Compound I relative to that prepared using crystalline Compound I, both solid dispersions still had significant levels of the inactive epimer.

In contrast, solid dispersion bulk powder prepared by spray drying had negligible levels of the inactive epimer of Compound I. Furthermore, samples of solid dispersion bulk powder prepared by spray drying had acceptable moisture contents and acceptable levels of Compound I degradation products following storage for 6 months at 5° C./ambient RH in a closed bottle, 25° C./60% relative humidity (RH)) in either an open or closed bottle, 30° C./ambient RH in a closed bottle, 40° C./75% RH in a closed bottle, or 50° C./ambient RH in a closed bottle.

Similarly, samples of exemplary pharmaceutical formulation G in tablets formed using solid dispersion prepared by spray drying that were stored in HDPE bottles following 1 month at 25° C./60% relative humidity (RH) in either an open or closed bottle, 40° C./75% RH in a closed bottle, or 50° C./ambient RH in a closed bottle had acceptable moisture contents between 0.74% and 3.7%, as compared to an initial moisture content of 2%. Likewise, samples stored under these conditions had acceptable label strength between 98.3 and 99.8% that was comparable to the initial label strength of 100%. Lastly, the presence of Compound I degradation products in samples stored under these conditions was comparable to that initially present. Based on the aforementioned results under various storage conditions, this pharmaceutical formulation exhibits desirable attributes and provides a commercially acceptable shelf-life projected to be at least 1 year under ambient conditions.

### Dissolution of Pharmaceutical Formulations

The dissolution of tablets prepared from the pharmaceutical formulations of the present invention detailed in Table 3A was determined with a USP Dissolution Apparatus II, using a paddle, operated at 75 rpm. A sample dissolution profile was obtained in 900 ml dissolution medium at pH 3.5. The dissolution medium contained 0.5% sodium lauryl sulfate in 0.05% acetic acid maintained at 37° C. The samples were analyzed by a reverse phase HPLC system using an)(Bridge 15 μC18, 4.6×50 mm column maintained at 30° C. Isocratic elution was used with a mobile phase consisting of 0.05% ammonium hydroxide:methanol (30:70 v/v) with a 1.5 ml/minute flow rate. An external standard method of quantitation was used with UV detection at 220 nm.

All pharmaceutical formulations detailed in Table 3A showed comparable dissolution profiles as illustrated in Table 4. Specifically, dissolution was complete by 30 minutes. Consequently, these formulations provide an immediate release dissolution profile for Compound I.

TABLE 4
Average Percentage ± Standard Deviation Compound I released from exemplary pharmaceutical formulations

| Time | Exemplary Pharmaceutical Formulation Tested | | | | | | | | |
|---|---|---|---|---|---|---|---|---|---|
| (min) | G | H | I | J | K | L | M | N | O |
| 15 | 81 ± 2.0 | 76 ± 0.5 | 76 ± 0.4 | 82 ± 1.2 | 78 ± 1.7 | 85 ± 0.9 | 81 ± 1.5 | 78 ± 0.8 | 82 ± 1.7 |
| 30 | 101 ± 1.2 | 101 ± 0.5 | 101 ± 1.2 | 101 ± 0.5 | 102 ± 0.3 | 102 ± 1.0 | 100 ± 0.3 | 100 ± 0.5 | 101 ± 1.2 |

Similarly, samples of exemplary pharmaceutical formulation G in tablets formed using solid dispersion prepared by spray drying that were assayed initially or following 1 month storage in HDPE bottles at 40° C./75% RH in a closed bottle or 50° C./ambient RH in a closed bottle all had comparable dissolution profiles with 80% dissolution at 20 minutes and greater than 95% dissolution at 45 minutes. This pharmaceutical formulation retains an immediate release dissolution profile for Compound I even after storage under accelerated conditions and so would be expected to exhibit the same dissolution profile following storage under ambient conditions for at least 1 year.

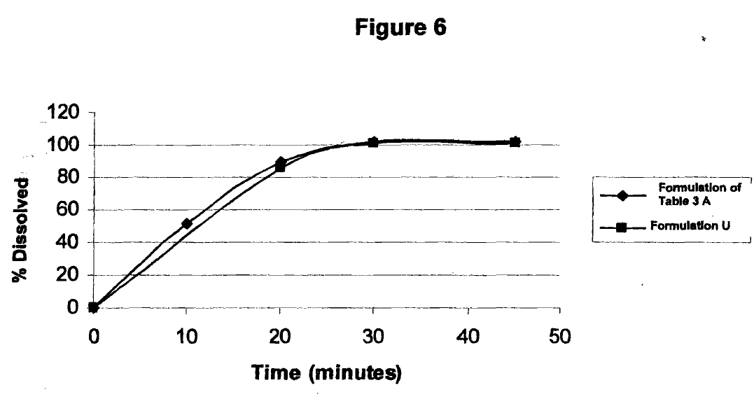

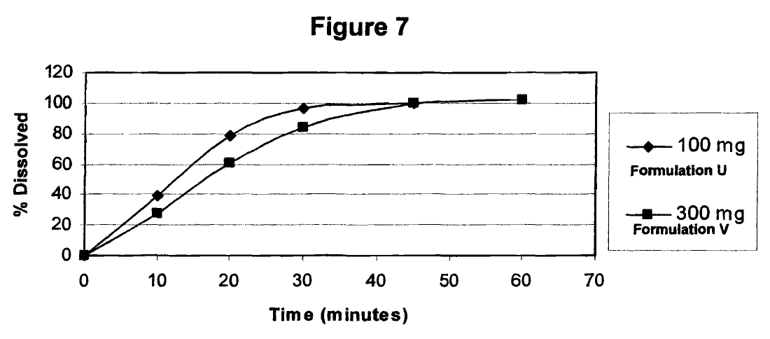

The in vitro dissolution profiles of tablets of an exemplary formulation G of Table 3A and of exemplary formulation U (Table 3 C) were determined using the USP Dissolution Apparatus II, as described above herein. Results obtained are graphically illustrated in FIG. 6. Dissolution of Tablets of both exemplary formulations was complete by 30 minutes. The in vitro dissolution profiles of tablets of exemplary formulations U and V (Table 3C) were similarly determined and results are graphically illustrated in FIG. 7. As shown in FIG. 7, dissolution of tablets containing 100 mg of Compound I (Formulation U) occurred more quickly than tablets containing 300 mg of Compound I (Formulation V). Dissolution of the latter tablets was complete by 40 minutes.

### Example 2

### Bioavailability of Pharmaceutical Formulations

Pharmaceutical formulations comprising a solid molecular dispersion of Compound I and at least one polymer were administered to dogs to assess bioavailability. In order to evaluate whether the bioavailability of Compound I when administered in a solid molecular dispersion of the invention was enhanced relative to comparator pharmaceutical formulations of Compound I lacking such a solid dispersion (specifically, a self-emulsifying drug delivery system (SEDDS) (No. 1 of Table 5A), amorphous formulation (No. 2 of Table 5A), and a micronized formulation) (MC, No. 3 of Table 5A), the following experiments were conducted. The specific comparator formulations examined are summarized in Tables 5A and designated formulations 1-3 and in Table 5B designated formulation 8. The formulations of the invention, designated R and S are summarized in Table 5A, and designated T in tablet and capsule forms and F in tablet form are summarized in Table 5B.

The SEDDS formulation (i.e., No. 1 in Table 5A) was prepared as follows. Firstly, Cremophor RH 40 (pplyoxy 40 hydrogenated castor oil), propylene glycol, and Capryol 90 (propylene glycol monocaprylate), were mixed at 60° C. until a clear solution was obtained in a ratio by weight of about 2.9 to 1 to about 4.9, respectively. Secondly, after the clear solution cooled, 400 mg Compound I was dissolved in 4 g of this solution followed by addition of 20 ml water. The resultant solution was encapsulated at a unit dose of 100 mg Compound I. Notably, the SEDDS formulation is stable for a period of 24 hours after reconstitution when stored at 2° C. to 8° C. and so was used within that time period.

The amorphous and micronized crystalline formulations of Compound I (i.e., No. 2 and 3, respectively, in Table 5A) were prepared by blending Compound I in either an amorphous or micronized crystalline form, respectively, with sodium lauryl sulphate in a ratio by weight of about 7 to 1. The resultant blend was encapsulated at a unit dose of 200 mg Compound I for the amorphous formulation and at a unit dose of 50 mg Compound I for the micronized crystalline formulation.

The formulations according to the present invention were prepared as described above in Example 1.

TABLE 5A
200 mg dose of formulations evaluated in dogs

| | | Unit | Unit Formulation | | |
|---|---|---|---|---|---|
| | Formulation | (Compound | | Amount | Amount |
| No. | Description | I) | Ingredient | (%) | (mg) |
| 1 | Self- | Capsule | Compound I | 16.62 | 100 |
| | Emulsifying | (100 mg) | Cremophor ® RH 40 | 27.60 | 166 |
| | Drug | | Propylene Glycol | 9.49 | 57.1 |
| | Delivery | | Capryol ™ 90 | 46.29 | 278 |
| | System | | Total | 100 | 601 |
| | (SEDDS) | | | | |
| 2 | Amorphous | Capsule | Compound I | 86.96 | 200 |
| | (Amorph) | (200 mg) | Sodium Lauryl | 13.04 | 30.0 |
| | | | Sulphate (SLS) | | |
| | | | Total | 100 | 230 |
| 3 | Micronized | Capsule | Compound I | 86.96 | 100 |
| | Crystalline | (100 mg) | Sodium Lauryl | 13.04 | 15.0 |
| | | | Sulphate (SLS) | | |
| | (MC) | | Total | 100 | 115 |
| R | 50%-loading | Capsule | Compound I | 36.78 | 100 |
| | Solid | (100 mg) | Copovidone | 36.78 | 100 |
| | Dispersion^{SDP} | | Crospovidone | 3.68 | 10.0 |
| | (SD), | | SLS | 3.68 | 10.0 |
| | Exemplary | | Silicon Dioxide | 0.46 | 1.25 |
| | Formulation | | Magnesium Stearate | 0.23 | 0.63 |
| | R | | Pregelatinized Starch | 18.39 | 50.0 |
| | | | Total | 100 | 272 |
| S | 25%-loading | Capsule | Compound I | 20.67 | 50.0 |
| | Solid | (50 mg) | Copovidone | 62.01 | 150 |
| | Dispersion^{SDP} | | Crospovidone | 4.13 | 10.0 |
| | (SD), | | SLS | 2.07 | 5.00 |
| | Exemplary | | Silicon Dioxide | 0.52 | 1.25 |
| | Formulation | | Magnesium Stearate | 0.26 | 0.63 |
| | S | | Pregelatinized Starch | 10.34 | 25.0 |
| | | | Total | 100 | 242 |

^{SDP}Solid Dispersion prepared by spray drying process.

TABLE 5B
400 mg dose of formulations evaluated in dogs

| | | Unit | Unit Formulation | | |
|---|---|---|---|---|---|
| | Formulation | (Compound | | Amount | Amount |
| No. | Description | I) | Ingredient | (%) | (mg) |
| T | Solid | Tablet | Compound I | 31.14 | 400 |
| | Dispersion^{SDP}, | (400 mg) | Copovidone | 31.14 | 400 |
| | Exemplary | | SLS | 3.11 | 40.0 |
| | Formulation T | | Sodium | 18.69 | 240 |
| | | | Croscarmellose | | |
| | | | Microcrystalline | 7.79 | 100 |
| | | | Cellulose | | |
| | | | Pregelatinized Starch | 7.79 | 100 |
| | | | Silicon Dioxide | 0.17 | 2.20 |
| | | | Magnesium Stearate | 0.17 | 2.20 |
| | | | Total | 100 | 1284 |
| F | Solid | Tablet | Compound I | 30.20 | 400 |
| | Dispersion^{HME} | (400 mg) | Copovidone | 30.20 | 400 |
| | Exemplary | | Triethyl Citrate | 3.02 | 40.0 |
| | Formulation F | | SLS | 3.02 | 40.0 |
| | | | Sodium | 25.67 | 340 |
| | | | Croscarmellose | | |
| | | | Pregelatinized Starch | 7.55 | 100 |
| | | | Silicon Dioxide | 0.17 | 2.20 |
| | | | Magnesium Stearate | 0.17 | 2.20 |
| | | | Total | 100 | 1324 |
| 8 | Amorphous | Capsule | Compound I | 90.91 | 200 |
| | (Amorph) | (200 mg) | SLS | 9.09 | 20.0 |
| | | | Total | 100 | 220 |
| T | Solid | Capsule | Compound I | 31.14 | 133 |
| | Dispersion^{SDP}, | (133 mg) | Copovidone | 31.14 | 133 |
| | Exemplary | | SLS | 3.11 | 13.3 |
| | Formulation T | | Sodium | 18.69 | 80.0 |
| | | | Croscarmellose | | |
| | | | Microcrystalline | 7.79 | 33.3 |
| | | | Cellulose | | |
| | | | Pregelatinized Starch | 7.79 | 33.3 |
| | | | Silicon Dioxide | 0.17 | 0.73 |
| | | | Magnesium Stearate | 0.17 | 0.73 |
| | | | Total | 100 | 428 |

^{SDP}Solid Dispersion prepared by spray drying process.
^{HME}Solid Dispersion prepared by hot melt extrusion process.

Fasted male beagle dogs received a single oral dose of 200 mg Compound I, administered in one of five different formulations detailed in Table 5A. Similarly, fasted male beagle dogs received a single oral dose of 400 mg Compound I, administered in one of four different formulations detailed in Table 5B. Plasma samples from each dog were analyzed for Compound I using liquid chromatography-tandem mass spectrometry (LC-MS/MS). In brief, samples were prepared using a protein precipitation extraction method. Extracts were analyzed in a PE Sciex API 5000 Tandem Mass Spectrometer equipped with a heated nebulizer source. Ions were detected using multiple reaction monitoring mode.

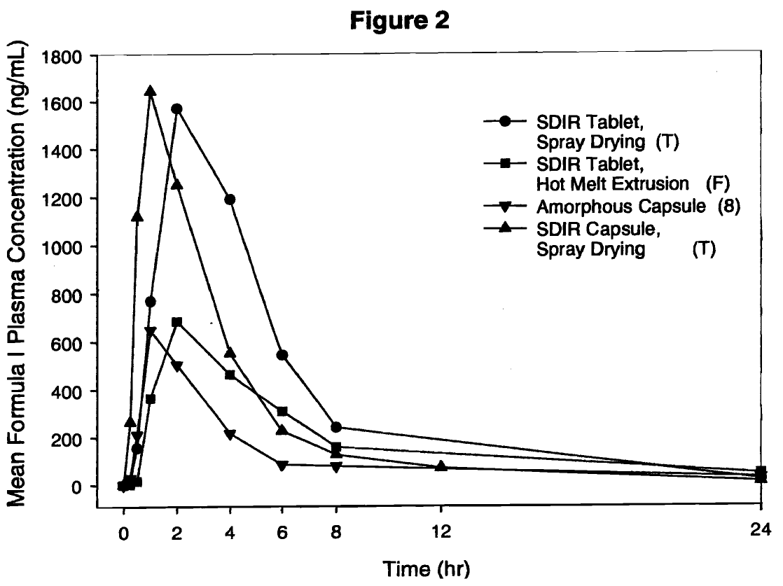

The pharmacokinetic profiles of Compound I following a single oral administration of 200 mg or 400 mg Compound I are illustrated in FIGS. 1 and 2, respectively. Likewise, the resultant pharmacokinetic parameters for Compound I following administration of the various formulations comprising 200 mg or 400 mg Compound I are summarized in Tables 6A and 6B, respectively.

TABLE 6A
Mean (CV) Compound I Plasma Pharmacokinetic Parameters Following
a Single Oral Dose of 200 mg Compound I, Administered in Five
Different Capsule Formulations, to Fasted Male Beagle Dogs

| | 1 | | 2 | | | | R | | S | |
|---|---|---|---|---|---|---|---|---|---|---|
| | SEDDS | | Amorphous | | 3 | | 50%-loading | | 25%-loading | |
| | Capsule | | Capsule | | MC Capsule | | SD Capsule | | SD Capsule | |
| | (2 × 100 mg) | | (1 × 200 mg) | | (2 × 100 mg) | | (2 × 100 mg) | | (4 × 50 mg) | |
| Parameter, | Mean | | Mean | | Mean | | Mean | | Mean | |
| (Unit) | (n = 6) | (CV) | (n = 6) | (CV) | (n = 6) | (CV) | (n = 6) | (CV) | (n = 6) | (CV) |
| Dog Wt., (kg) | 10.4 | (11) | 10.7 | (12) | 10.4 | (12) | 10.4 | (11) | 10.5 | (11) |
| Dose, (mg/kg) | 19.5 | (11) | 18.9 | (12) | 19.5 | (13) | 19.4 | (12) | 19.3 | (12) |
| Cmax, (ng/mL) | 5028 | (21) | 498 | (56) | 128 | (53) | 1864 | (72) | 2333 | (51) |
| Tmax, (hr) | 0.917 | (22) | 1.00 | (0) | 1.04 | (53) | 1.67 | (31) | 1.67 | (31) |
| AUC(0-8 hr), | 13077 | (30) | 1604 | (46) | 427 | NC | 4736 | (69) | 6959 | (53) |
| (ng · hr/mL) | | | | | | | | | | |
| AUC(tf), | 13954 | (29) | 1363 | (65) | 333 | (31) | 5393 | (70) | 8026 | (63) |
| (ng · hr/mL) | | | | | | | | | | |
| tf, (hr) | 13.3 | (62) | 7.00 | (35) | 5.33 | (43) | 10.7 | (61) | 13.3 | (62) |
| Cmax/Dose | 263 | (27) | 27.7 | (62) | 6.89 | (62) | 99.8 | (72) | 125 | (56) |
| AUC(tf)/Dose | 730 | (34) | 75.4 | (68) | 18.1 | (41) | 291 | (73) | 431 | (68) |

Following a single oral dose of the formulations 200 mg Compound I to fasted male beagle dogs the five formulations evaluated were ranked as follows:
Mean Compound I Cmax values: SEDDS capsule (1)>25%-loading SD capsule (S)>50%-loading SD capsule (R)>Amorphous capsule (2)>Micronized Crystalline capsule (3).
Mean Compound I Tmax values: 25%-loading SD capsule (S)=50%-loading SD capsule (R)>Micronized Crystalline capsule (3)>Amorphous capsule (2)>SEDDS capsule (1).
Mean Compound I exposure values: SEDDS capsule (1)>25%-loading SD capsule (S)>50%-loading SD capsule (R)>Amorphous capsule (2)>Micronized Crystalline capsule (3).
   Although the SEDDS formulation provides the greatest Cmax and exposure values per dose of the formulations examined, the SEDDS formulation is not practical for commercialization as it is only stable for a period of 24 hours after reconstitution when stored at 2° C. to 8° C. In contrast, the 25%-loading and 50%-loading solid molecular dispersion formulations according to the present invention not only provide significantly higher bioavailability in dogs compared with either amorphous or crystalline comparator formulations of Compound I but also surprisingly maintain Compound I in an amorphous form that is stable within the solid dispersions for a commercially acceptable shelf-life of at least 1 year under ambient conditions.

TABLE 6B
Mean (CV) Compound I Plasma Pharmacokinetic Parameters Following
a Single Oral Dose of 400 mg Compound I, Administered in Four Different
Tablet or Capsule Formulations, to Fasted Male Beagle Dogs

| | T | | F | | 8 | | T | |
|---|---|---|---|---|---|---|---|---|
| | SD Tablet SDP | | SD Tablet HME | | Amorph Capsule | | SD Capsule SDP | |
| | (1 × 400 mg) | | (1 × 400 mg) | | (2 × 200 mg) | | (3 × 133 mg) | |
| | Mean^{a} | | Mean^{a} | | Mean | | Mean | |
| Parameter, (Unit) | (n = 5) | (CV) | (n = 5) | (CV) | (n = 5) | (CV) | (n = 6) | (CV) |
| Dog Wt., (kg) | 11.5 | (8) | 11.1 | (15) | 11.0 | (14) | 8.65 | (13) |
| Dose, (mg/kg) | 35.1 | (8) | 36.6 | (14) | 36.9 | (15) | 46.9 | (12) |
| Cmax, (ng/mL) | 1751 | (61) | 863 | (80) | 692 | (52) | 1791 | (61) |
| Tmax, (hr) | 2.40 | (37) | 2.40 | (37) | 1.80 | (72) | 1.08 | (45) |
| AUC(0-8 hr), | 6704 | (66) | 3007 | (87) | 2015 | (44) | 5275 | (47) |
| (ng · hr/mL) | | | | | | | | |
| AUC(tf), | 8782 | (69) | 5103 | (95) | 3243 | (64) | 6138 | (44) |
| (ng · hr/mL) | | | | | | | | |
| tf, (hr) | 24.0 | (0) | 30.4 | (57) | 33.6 | (39) | 24.0 | (0) |
| Cmax/Dose | 50.1 | (62) | 23.1 | (83) | 18.2 | (49) | 39.2 | (65) |
| AUC(tf)/Dose | 252 | (70) | 135 | (97) | 87.2 | (64) | 134 | (49) |

^{a}Dog No. 11 was excluded from the calculation of means due to anomalous terminal-phase concentrations

Following a single oral dose of 400 mg Compound I to fasted male beagle dogs, the four formulations evaluated were ranked as follows:
Mean Compound I Cmax values: SD tablet (spray drying) (T)>SD capsule (spray drying) (T)>SD tablet (hot melt extrusion) (F)>Amorphous capsule (8).
Mean Compound I Tmax values: SD tablet (spray drying) (T)=SD tablet (hot melt extrusion)(F)>Amorphous capsule (8)>SD capsule (spray drying)(T).
Mean Compound I exposure values: SD tablet (spray drying) (T)>SD tablet (hot melt extrusion)(F)≈SDIR capsule (spray drying)(T)>Amorphous capsule (8).
   Interestingly, although Cmax and exposure following oral administration of the Amorphous capsules increased with dose over the range of 200 to 400 mg, the dose-adjusted Cmax values tended to be lower following the administration of 400 mg Amorphous capsule compared to a similar formulation at 200 mg.

Strikingly, despite significant epimerization of Compound I in solid dispersions prepared by the hot melt extrusion process, enhanced bioavailability of Compound I was obtained with pharmaceutical formulations comprising solid dispersions prepared either by hot melt extrusion or spray drying relative to the comparator Amorphous formulation (8) of Compound I. Both Cmax and exposure of the SD tablet prepared by hot melt extrusion was at least 1.5 times greater than that obtained with Amorphous capsules. Likewise, both Cmax and exposure of the SD capsule and tablet prepared by spray drying was at least 1.5 times greater than that obtained with Amorphous capsules. Surprisingly, Cmax and exposure of the SD tablet prepared by spray drying was about 2 times greater than that obtained with the SD tablet prepared by hot melt extrusion process. This result was unexpected as both solid dispersions utilized the same polymer in the same ratio of Compound I to polymer.

### Example 3

### Clinical Study 1

### Pharmacokinetic Profile of Compound I Administered in a Formulation of the Present Invention (Exemplary Formulation G) in a Dosage Form as Capsule or Tablet, in Comparison with a Comparator Formulation, i.e., a Suspension Under Fed and Fasted Conditions in Healthy Volunteers

The pharmacokinetic profile of Compound I after administration in each of three different formulations (i.e., capsule or tablet dosage form of the present invention, or as a comparative example, a suspension i.e. not within the prevent invention) was ascertained in healthy volunteers under either fed or fasted conditions. Specifically, healthy volunteers were administered a single oral dose of a formulation G (Table 3A above) comprising 200 mg Compound I (2×100 mg capsule; 2×100 mg tablet); or a comparator formulation comprising 200 mg Compound as 20 ml of 10 mg/mL suspension) under either fed conditions (i.e., following a standard meal) or fasted conditions (i.e., following an overnight fast). Specifically, subjects received either capsules or tablets of exemplary formulation G described in Table 3A. As a comparator, subjects received a comparative suspension formulation, prepared by suspending 200 mg Compound I in 20 ml solution of Ora-Sweet SF™ (a commercially available vehicle from Paddock Laboratories, Inc., Minneapolis, Minn., that mainly contains 10% sorbitol, 9% glycerine, and 0.1% sodium saccharin) and 0.25% sodium lauryl sulfate. Blood was collected from each subject pre-dose, as well as 0.5, 1, 1.5, 2, 3, 4, 5, 6, 7, 8, 10, 12, 16, and 24 hr post-dose for determining the concentration of Compound I in the plasma and to calculate the pharmacokinetic parameters for each formulation.

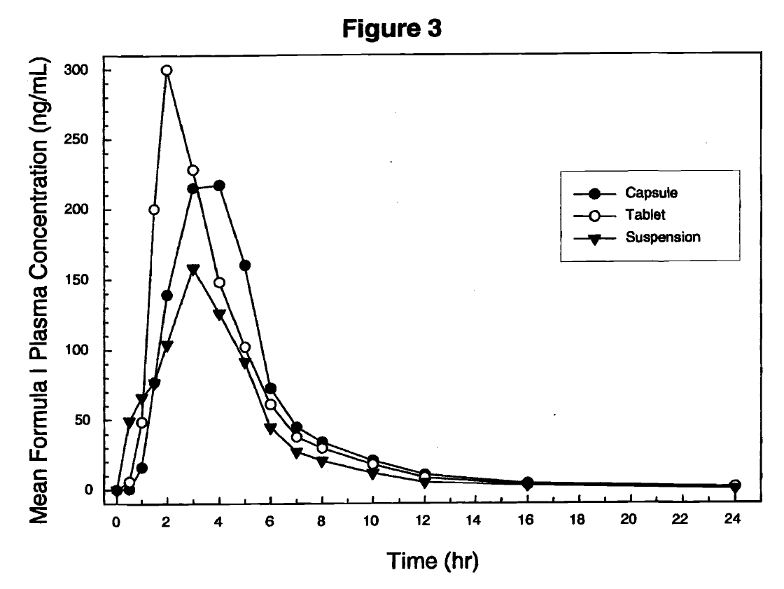

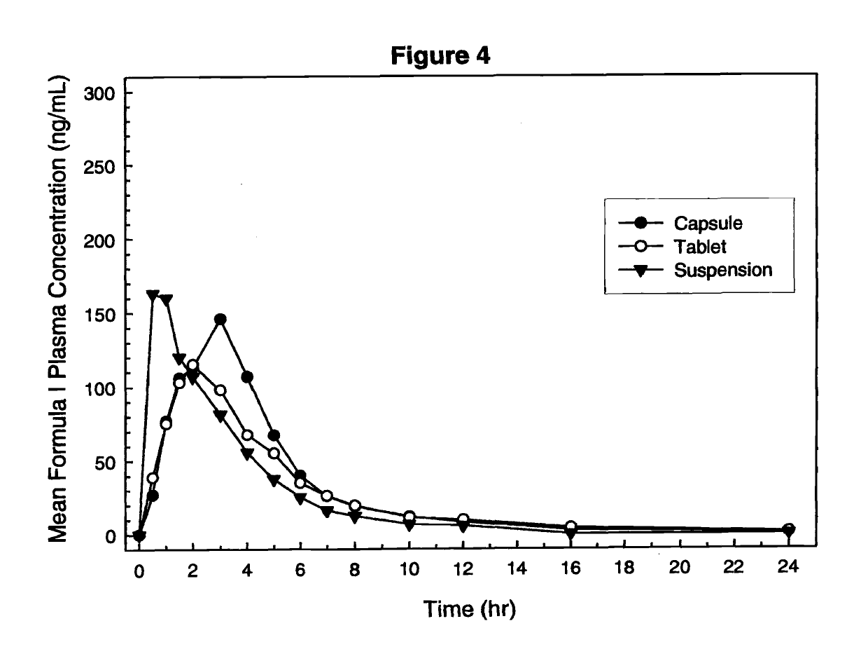

The resulting mean plasma concentration/time profile for Compound I capsule, tablet, or suspension formulations under fed conditions is displayed graphically in FIG. 3. Similarly, the resulting mean plasma concentration/time profile for Compound I capsule, tablet, or suspension formulations under fasted conditions is displayed graphically in FIG. 4. Likewise, the mean (coefficient of variation (CV), %) as well as range for pharmacokinetic parameters of Compound I, specifically, Tmax, Cmax and exposure (AUC(1)), for each formulation of Compound I examined under fed and fasted conditions is summarized in Table 7 below.

TABLE 7
Mean (*CV, %) and Range for Pharmacokinetic Parameters of
Compound I in Healthy Human Subjects after Single Dose

| | | | Tmax (hr) ^{a} | Cmax (ng/mL) | AUC(l) (hr-ng/mL) |
|---|---|---|---|---|---|
| Food | Formulation | n | [Range] | [Range] | [Range] |
| Fed | G | 12 | 3.5 | 316 (69%) | 1090 (54%) ^{b} |
| | Capsule | | [2-5] | [109-910] | [540-2540] |
| Fed | G | 12 | 2 | 350 (67%) | 1120 (54%) ^{b} |
| | Tablet | | [1.5-5] | [104-944] | [600-2450] |
| Fed | Suspension | 12 | 3 | 191 (73%) | 767 (40%) ^{c} |
| | | | [0.5-5] | [86.6-599] | [329-1870] |
| Fasted | G | 12 | 3 | 177 (44%) | 732 (40%) ^{b} |
| | Capsule | | [1-4] | [51.8-291] | [267-1220] |
| Fasted | G | 12 | 2 | 127 (30%) | 616 (33%) ^{b} |
| | Tablet | | [1-3] | [61.2-184] | [253-851] |
| Fasted | Suspension | 12 | 1 | 183 (27%) | 604 (28%) ^{b} |
| | | | [0.5-2] | [116-252] | [224-773] |

*CV = the coefficient of variation defined as the ratio of the standard deviation to the mean.
^{a} Tmax presented as median [range].
^{b} 1 subject excluded and
^{c} 2 subjects excluded due to R² <0.90 at terminal phase.

Food increased the relative oral bioavailability of Compound I. In particular, the relative oral bioavailability of Compound I in healthy human subjects under fed conditions compared with that under fasted conditions was 149% for the capsule dosage form and 182% for the tablet dosage form of formulations according to the present invention. In contrast, relative oral bioavailability of a comparator formulation was 127% for the suspension. Thus, Compound I is preferably administered with food. Furthermore, solid molecular dispersion formulations according to the present invention in capsule and tablet dosage forms increased Compound I exposure when compared to the comparator amorphous suspension formulation. In fact, the difference in AUC(1) was about 40 to 50% higher for capsule and tablet dosage forms of the present invention compared to the amorphous suspension formulation when the dose was administered under fed conditions (i.e., following a standard meal).

Clinical Study 2—Pharmacokinetic Profile of Compound I Following Once-a-Day Oral Administration of 300 mg Compound I in a Formulation of the Present Invention in Tablet Dosage Form in Combination with 100 mg Ritonavir for 10-Days to Healthy Human Subjects Under Fed Conditions

The pharmacokinetic profile of Compound I following once-a-day oral administration of 300 mg Compound I in a formulation of the present invention in tablet form (specifically exemplary formulation G described in Table 3A, 3×100 mg, QD) in combination with 100 mg ritonavir (RTV, 1×100 mg, QD) for 10-days to healthy human subjects under fed conditions (i.e., following a standard meal) was determined. Notably, steady-state levels of Compound I were achieved after 10-day dosing. Blood was collected from each subject pre-dose (on Days 7, 8, and 9), as well as 0.5, 1, 1.5, 2, 3, 4, 5, 6, 7, 8, 10, 12, 16, and 24 hr post-dose (on Day 10) for determining the concentration of Compound I in the plasma and to calculate the pharmacokinetic parameters for each formulation.

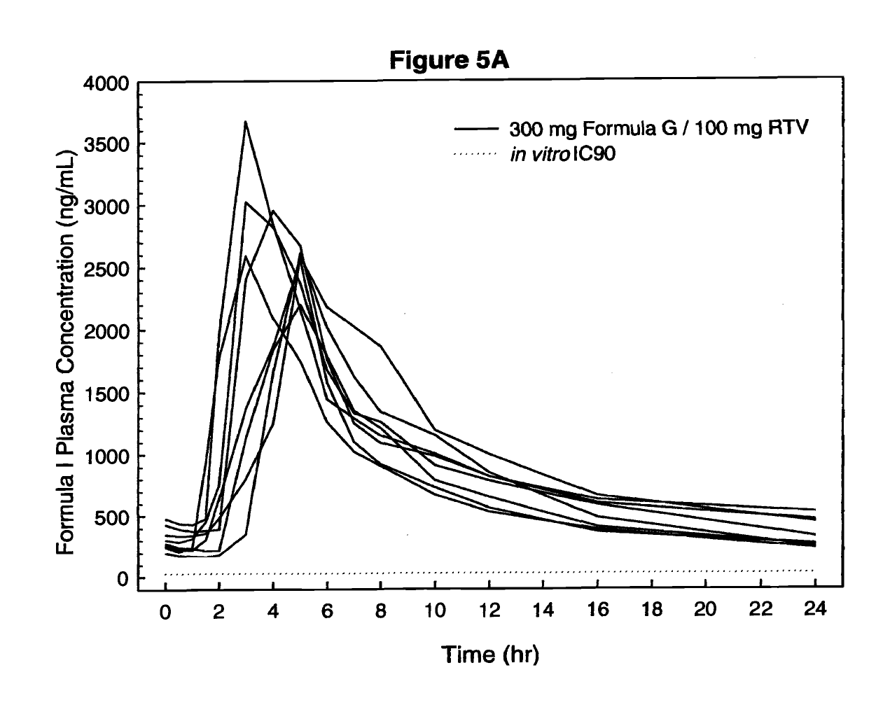

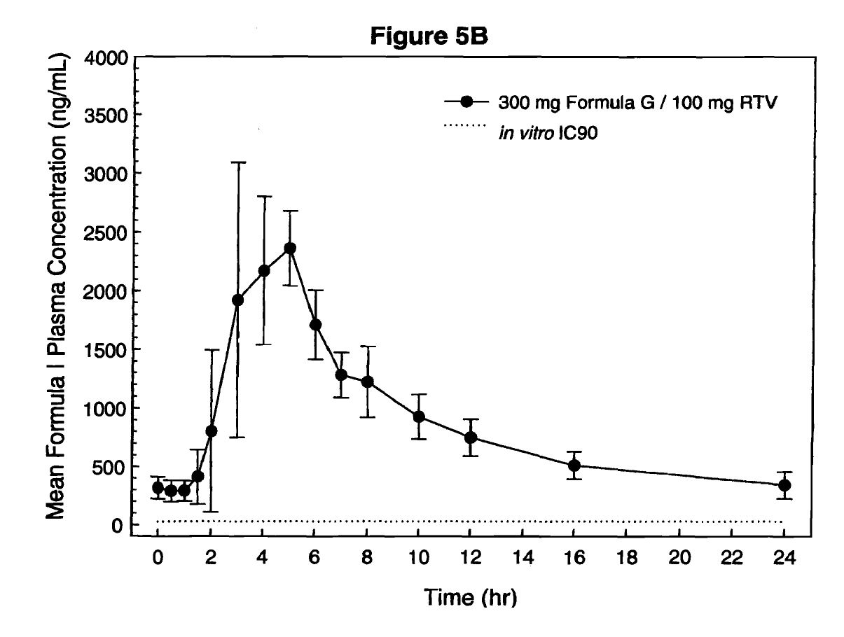

The resulting plasma concentration/time profiles of Compound I in eight individual healthy human subjects following once-a-day oral administration of the formulation of the invention containing 300 mg Compound I and 100 mg ritonavir for 10-days to the subjects under fed conditions is displayed graphically in FIG. 5A. Similarly, the resulting mean plasma concentration/time profiles and error bars of these same eight subjects are displayed graphically in FIG. 5B. For reference, the in vitro IC90 (28 ng/mL) of Compound I is also illustrated graphically in FIGS. 5A and 5B (see . . . ). The mean (coefficient of variation (CV)) as well as range for pharmacokinetic parameters of Compound I, specifically, Cmax, Cmin, and exposure (AUC(tau)), following once-a-day oral administration of the formulation of the invention containing 300 mg Compound I and 100 mg ritonavir for 10-days to healthy human subjects under fed conditions is summarized in Table 8 below.

TABLE 8
Mean (CV) and Range for Pharmacokinetic Parameters of Compound
I in a formulation of the invention in 8 Healthy Human Subjects
After 10-Day Dosing with Cytochrome P450 Inhibitor

| | Cmax | Cmin | AUC(tau) |
|---|---|---|---|
| | (ng/mL) | (ng/mL) ^{a} | (hr-ng/mL) |
| Mean | 2770 (16) | 280 (32) | 21000 (16) |
| (CV) | | | |
| Range | 2200-3670 | 167-426 | 17100-25700 |

^{a} Cmin = minimum observed concentration during the dosing interval

The minimum observed concentration (Cmin) range of Compound I (167-426 ng/mL) for these 8 subjects was at least 6 times higher than the in vitro IC90 (28 ng/mL) of Compound I. Consequently, when given with a standard meal, once a day administration of a formulation of the present invention containing 300 mg Compound I in combination with 100 mg ritonavir provides sufficient bioavailability to be therapeutically effective.

The present invention is not to be limited in scope by the specific embodiments described herein. Indeed, various modifications of the invention in addition to those described herein will become apparent to those skilled in the art from the foregoing description. Such modifications are intended to fall within the scope of the appended claims.

1. A pharmaceutical formulation comprising (a) Compound I and (b) at least one excipient in a solid molecular dispersion wherein the pharmaceutical formulation provides a mean AUC of Compound I that is at least about 16800 hr-ng/ml when administered at a dose equivalent to 300 mg Compound I in combination with a cytochrome P450 inhibitor once-a-day to a human subject.

2. The pharmaceutical formulation of claim 1 which provides a mean Cmax of Compound I that is at least 2216 ng/ml.

3. A pharmaceutical formulation comprising (a) Compound I and (b) at least one excipient in a solid molecular dispersion wherein the pharmaceutical formulation provides a mean Cmin of Compound I that is at least 200 ng/ml when administered at a dose equivalent to 300 mg Compound I in combination with a cytochrome P450 inhibitor once-a-day to a human subject.

4. The pharmaceutical formulation of claim 1 wherein the cytochrome P450 inhibitor is a cytochrome P450 isoenzyme 3A4 inhibitor.

5. The pharmaceutical formulation of claim 1 wherein the cytochrome P450 inhibitor is ritonavir.

6. The pharmaceutical formulation of claim 1 wherein the excipient is a non-swellable polymer.

7. A pharmaceutical formulation comprising:
(a) Compound I; and
(b) at least one excipient;
wherein (a) and (b) are in a solid molecular dispersion.

8. The pharmaceutical formulation of claim 7, wherein the excipient is at least one non-swellable polymer which is carbomer, cellulose acetate phthalate, hydroxypropyl cellulose, hydroexyethyl cellulose, hydroxypropylmethlycellulose, hydroxypropyl methylcellulose phthalate, polyacrylate polymate, polyethylene oxide, polyvinyl alcohol, poloxamer, povidone, polytheylene glycol, copovidone, or hypromellose acetate succinate, or a combination of two or more thereof.

9. The pharmaceutical formulation of claim 8, wherein at least one polymer is poloxamer, povidone, polytheylene glycol, copovidone, hydroxypolymethylcellulose, or hypromellose acetate succinate, or a combination of two or more thereof.

10. The pharmaceutical formulation of claim 7, wherein the excipient is at least one polymer which is copovidone.

11. The pharmaceutical formulation of claim 1, wherein the ratio by weight of (a) to (b) is in the range of about 10:1 to about 1:10.

12. The pharmaceutical formulation of claim 1, wherein the ratio by weight of (a) to (b) is in the range of about 1:1 to about 1:3.

13. The pharmaceutical formulation of claim 1, wherein the ratio by weight of (a) to (b) is about 1:1.

14. The pharmaceutical formulation of claim 1, wherein the ratio by weight of (a) to (b) is about 1:3.

15-16. (canceled)

17. The pharmaceutical formulation of claim 1, further comprising stearic acid, magnesium stearate, calcium stearate, fat, wax, hydrogenated vegetable oil, castor oil, glycerin monostearate, glyceryl behenate, sodium stearyl fumurate, zinc stearate, glyceryl palmitostearate, medium-chain triglyceride, or mineral oil, or a combination of two or more thereof.

18. The pharmaceutical formulation of claim 1, further comprising a diluent, a disintegrant, a surfactact, a glidant, a lubricant, or a combination of two or more thereof.

19. The pharmaceutical formulation of claim 1, wherein Compound I in an amorphous form is stable within the solid molecular dispersion after storage at 40° C. and 75% relative humidity for at least 3 months.

20. The pharmaceutical formulation of claim 1, which provides release of at least about 75% Compound I in 45 minutes when tested using a USP Dissolution Apparatus II with a paddle operated at 75 RPM filled with 900 mL of dissolution medium at pH 3.5 comprising 0.5% sodium lauryl sulfate in 0.05% acetic acid maintained at 37° C.±0.5° C.

21. The pharmaceutical formulation of claim 7, comprising:
(a) Compound I; and
(b) Crospovidone,
wherein (a) and (b) are in a solid molecular dispersion in which the ratio by weight of (a) to (b) is about 1:1 and the formulation further comprises sodium lauryl sulfate, sodium croscarmellose, silicon dioxide and magnesium stearate.

22. A method for treating or ameliorating one or more symptoms of HCV or disorders associated with HCV, comprising the step of administering to a patient in need thereof the pharmaceutical formulation of claim 7 comprising:
(a) Compound I; and
(b) at least one polymer;
wherein (a) and (b) are in a solid molecular dispersion.

23. (canceled)

24. A method for preparing a pharmaceutical formulation comprising Compound I in a solid dispersion with at least one polymer, comprising the steps of:
(c) dissolving Compound I or a solvate thereof and at least one polymer in an organic solvent; and
(d) evaporating the organic solvent to form a molecular dispersion of Compound I in amorphous form and said polymer.

25-27. (canceled)
